SEQUENCE LISTING (16 sequences)

SEQ ID NO: 1 - DNA, 24 bp, Artificial sequence
  Synthetic oligonucleotide.
cactacaggatgtttgtggacgtg

SEQ ID NO: 2 - DNA, 24 bp, Artificial sequence
  Synthetic oligonucleotide.
ccccttgttgtttgtgagctttag

SEQ ID NO: 3 - DNA, 24 bp, Artificial sequence
  Synthetic oligonucleotide.
tctggagctggcctttcatcatca

SEQ ID NO: 4 - DNA, 24 bp, Artificial sequence
  Synthetic oligonucleotide.
tctgctcacttccaaagagctggt

SEQ ID NO: 5 - DNA, 20 bp, Artificial sequence
  Synthetic oligonucleotide.
ggatgcagaaggagatcact

SEQ ID NO: 6 - DNA, 20 bp, Artificial sequence
  Synthetic oligonucleotide.
cgatccacacggagtacttg

SEQ ID NO: 7 - DNA, 17 bp, Artificial sequence
  Synthetic oligonucleotide.
gctagctgattaattaa

SEQ ID NO: 8 - protein, 17 aa, artificial sequence
  synthetic peptide
LEKNVTVTHSVNLLEDS

SEQ ID NO: 9 - protein, 17 aa, artificial sequence
  synthetic peptide
GNLIAPWYAFALSRGFG

SEQ ID NO: 10 - protein, 17 aa, artificial sequence
  synthetic peptide
WYAFALSRGFGSGIITS

SEQ ID NO: 11 - protein, 17 aa, artificial sequence
  synthetic peptide
SSLPFQNVHPVTIGECP

SEQ ID NO: 12 - protein, 17 aa, artificial sequence
  synthetic peptide
LEPGDTIIFEANGNLIA

SEQ ID NO: 13 - protein, 17 aa, artificial sequence
  synthetic peptide
SSFERFEIFPKESSWPN

SEQ ID NO: 14 - protein, 17 aa, artificial sequence
  synthetic peptide
EKEVLVLWGVHHPPNIG

SEQ ID NO: 15 - protein, 17 aa, artificial sequence
  synthetic peptide
VSVVSSHYSRRFTPEIA

SEQ ID NO: 16 - protein, 17 aa, artificial sequence
  synthetic peptide
HYSRRFTPEIAKRPKVR

(19) United States
(12) Patent Application Publication
(10) Pub. No.: US 2012/0128710 A1
(43) Pub. Date: May 24, 2012

(54) Enhancement of Pathogen-Specific Memory Th17 Cell Responses

(75) Inventors: SangKon Oh, Baltimore, MD (US); Jacques F. Banchereau, Montclair, NJ (US); Gerard Zurawski, Midlothian, TX (US); Hideki Ueno, Plano, TX (US); Ling Ni, Dallas, TX (US)

(73) Assignee: BAYLOR RESEARCH INSTITUTE, Dallas, TX (US)

(21) Appl. No.: 13/282,112

(22) Filed: Oct. 26, 2011

Related U.S. Application Data
(60) Provisional application No. 61/409,507, filed on Nov. 2, 2010.

(51) Int. Cl. A61K 39/395; A61P 31/16; C07K 19/00

(52) U.S. Cl. 424/192.1; 530/387.3

(57) ABSTRACT

Compositions and methods for enhancing Th1/Th17 cell responses and decreasing Th2 cell responses are disclosed herein. In various embodiments the present invention describes activation of human dendritic cells and enhancement of antigen-specific T cell responses in a Dectin-1-expressing human dendritic cells comprising an anti-Dectin-1-specific antibody or fragment thereof fused with one or more antigens. TLR2 ligands may also be included to enhance the activation and for enhancement of T-cell responses. Further, the invention also includes methods based on the compositions described herein for the treatment of pathogenic infections.

### CROSS-REFERENCE TO RELATED APPLICATIONS

This application claims the benefit of U.S. provisional patent application No. 61/409,507, filed Nov. 2, 2010 the contents of which are incorporated by reference herein.

### STATEMENT OF FEDERALLY FUNDED RESEARCH

This invention was made with U.S. Government support awarded by the National Institutes of Health (NIH) under Contract No. U19 AI057234. The government has certain rights in this invention.

### TECHNICAL FIELD OF THE INVENTION

The present invention relates in general to immunity against pathogens, and more particularly, to delivering antigens to human dendritic cells (DCs) via Dectin-1 to enhance pathogen-specific Th17 cells in memory pools.

### REFERENCE TO A SEQUENCE LISTING

A Sequence Listing is attached and incorporated herein. The Sequence Listing reflects the sequences set forth as originally filed with no new matter incorporated into the Specification.

### BACKGROUND OF THE INVENTION

Without limiting the scope of the invention, its background is described in connection with targeting antigens to enhance Th17 cells and immunity against pathogens. United States Patent Application No. 2010/0166784 (Murphy et al., 2010) describes a method to modulate the development of Th17 or Treg cells. The Murphy invention provides methods of modulating an immune response in a host by providing a nucleic acid sequence that modulates the development of Th17 or Treg cells.

United States Patent Application No. 2008/0233140 (Banchereau et al., 2008) includes compositions and methods for binding Dectin-1 on immune cells with anti-Dectin-1-specific antibodies or fragments thereof capable of activating the immune cells.

### SUMMARY OF THE INVENTION

The present invention describes compositions and methods for enhancing pathogen-specific T cell responses using human dendritic cells. The method describes an anti-Dectin-1-specific antibody or binding fragment thereof fused with one or more antigens, that may be used in the presence or absence of TLR2 ligands to enhance Th1 and Th17 cell responses and at the same time decrease Th2 cell responses. Methods for treating pathogenic infections using the compositions described herein are also presented that drive the immune response to a Th1 and Th17 helper T cells responses.

The instant invention in one embodiment provides a method for enhancing antigen-specific T cell responses in a Dectin-1-expressing antigen presenting cell (APC) comprising: (i) loading the APC with an anti-Dectin-1-specific antibody or binding fragment thereof conjugated or fused with one or more antigens, (ii) contacting the antigen-loaded APC with T cells, and (iii) isolating T cells that proliferate when contacted with the antigen-loaded APC wherein the antigen-specific T cell response is enhanced to secrete IL-23.

In one aspect of the method provided hereinabove the one or more antigens comprise bacterial, fungal or viral antigens. In a specific aspect of the method above the antigen is a HA1 subunit of an influenza virus. In another aspect the composition optionally comprises one or more TLR2 ligands. In another aspect the one or more TLR2 ligands comprise heat-killed bacteria, lipoglycans, lipopolysaccharide, lipoteichoic acids, peptidoglycans, synthetic lipoproteins, zymosan or combinations and modifications thereof. In yet another aspect the TLR2 ligand comprises lipopolysaccharides comprising P. gingivalis LPS or E. coli LPS. In a related aspect the method enhances Th17 and Th1 and reduces Th2 cell responses.

Another embodiment of the instant invention describes a method for enhancing antigen-specific T cell responses in a Dectin-1-expressing antigen presenting cell (APC) comprising the step of contacting the APC with an anti-Dectin-1-specific antibody or fragment thereof fused with one or more antigens and one or more TLR2 ligands. The one or more antigens of the method comprise bacterial, fungal or viral antigens. In one aspect the antigen is a HA1 subunit of an influenza virus. In another aspect the one or more TLR2 ligands comprise heat-killed bacteria, lipoglycans, lipopolysaccharide, lipoteichoic acids, peptidoglycans, synthetic lipoproteins, zymosan or combinations and modifications thereof. In yet another aspect the composition comprises lipopolysaccharides comprising P. gingivalis LPS or E. coli LPS. In another aspect the method increases secretion of IL-1β, IL-6, and IL-23 thereby leading to an enhanced Th17 response. In another aspect the method reduces Th2 cell responses.

In yet another embodiment the instant invention relates to an influenza vaccine composition for prophylaxis, treatment, amelioration of symptoms or combinations thereof comprising: an anti-Dectin-1-specific antibody or binding fragment thereof fused with a HA1 subunit of an influenza virus and one or more optional pharmaceutically acceptable excipients or adjuvants. In one aspect the composition optionally comprises one or more TLR2 ligands. In another aspect the one or more TLR2 ligands comprise heat-killed bacteria, lipoglycans, lipopolysaccharide, lipoteichoic acids, peptidoglycans, synthetic lipoproteins, zymosan or combinations and modifications thereof. The TLR2 ligands of the instant invention comprise lipopolysaccharides comprising P. gingivalis LPS or E. coli LPS. In one aspect the composition enhances Th17 and Th1 responses by a secretion of IL-23. In another aspect the composition reduces Th2 cell responses. In another aspect the composition is administered by an oral route, a parenteral route or an intra-nasal route.

An influenza vaccine composition for prophylaxis, treatment, amelioration of symptoms or combinations thereof is described in one embodiment of the present invention. The vaccine of the present invention comprises: an anti-Dectin-1-specific antibody or binding fragment thereof fused with a HA1 subunit of an influenza virus, one or more TLR2 ligands comprising P. gingivalis LPS or E. coli LPS or combinations thereof, and one or more optional pharmaceutically acceptable excipients or adjuvants. In one aspect the composition increases secretion of IL-1β, IL-6, and IL-23 thereby leading to an enhanced Th17 response and reduces Th2 cell responses. The composition of the present invention is administered by an oral route, a parenteral route or an intra-nasal route.

Another embodiment of the instant invention discloses a method for treating, prophylaxis or amelioration of symptoms of influenza in a human subject comprising the steps of: identifying the subject in need of the treatment, prophylaxis or amelioration of symptoms of the influenza and administering a therapeutically effective amount of a pharmaceutical composition or a vaccine comprising an anti-Dectin-1-specific antibody or binding fragment thereof fused with a HA1 subunit of an influenza virus and one or more optional pharmaceutically acceptable excipients or adjuvants in an amount sufficient for the treatment, prophylaxis or amelioration of the symptoms of the influenza. In one aspect the composition is administered by an oral route, a parenteral route or an intra-nasal route.

In yet another embodiment the present invention discloses a method for treating, prophylaxis or amelioration of symptoms of influenza in a human subject comprising the steps of: identifying the subject in need of the treatment, prophylaxis or amelioration of symptoms of the influenza and administering a therapeutically effective amount of a pharmaceutical composition or a vaccine comprising an anti-Dectin-1-specific antibody or binding fragment thereof fused with a HA1 subunit of an influenza virus, TLR2 ligands comprising P. gingivalis LPS or E. coli LPS or combinations thereof, and one or more optional pharmaceutically acceptable excipients or adjuvants in an amount sufficient for the treatment, prophylaxis or amelioration of the symptoms of the influenza. In one aspect of the method described above the composition is administered by an oral route, a parenteral route or an intra-nasal route.

One embodiment of the present invention relates to a composition for enhancing antigen-specific T cell responses in a Dectin-1-expressing antigen presenting cell (APC) comprising an anti-Dectin-1-specific antibody or binding fragment thereof fused with one or more antigens. The APC of the present invention comprises an isolated dendritic cell (DC), a peripheral blood mononuclear cell (PBMC), a monocyte, a B cell, a myeloid dendritic cell or combinations thereof. In one aspect the APC comprises an isolated dendritic cell (DC), a peripheral blood mononuclear cell, a monocyte, a B cell, a myeloid dendritic cell or combinations thereof that have been cultured in vitro with GM-CSF and IL-4, IFNα, antigen, and combinations thereof. In another aspect the one or more antigens comprises bacterial, fungal or viral antigens, wherein the antigen is a HA1. subunit of an influenza virus and optionally comprises one or more TLR2 ligands. In one aspect the one or more TLR2 ligands comprise heat-killed bacteria, lipoglycans, lipopolysaccharide, lipoteichoic acids, peptidoglycans, synthetic lipoproteins, zymosan or combinations and modifications thereof. In another aspect the TLR2 ligand comprises lipopolysaccharides comprising P. gingivalis LPS or E. coli LPS. In another aspect the composition results in a proliferation of CD4⁺ T cells. In yet another aspect the CD4⁺ T secrete one or more cytokines selected from the group consisting of IFNγ, IL-13, IL-10, IL-17, and IL-21. In one aspect the composition enhances Th17 and Th1 responses by a secretion of IL-23. In another aspect the composition reduces Th2 cell responses.

In another embodiment the instant invention presents a composition for enhancing antigen-specific T cell responses in a Dectin-1-expressing antigen presenting cell (APC) comprising an anti-Dectin-1-specific antibody or binding fragment thereof fused with one or more antigens and one or more TLR2 ligands. In one aspect the APC comprises an isolated dendritic cell (DC), a peripheral blood mononuclear cell (PBMC), a monocyte, a B cell, a myeloid dendritic cell or combinations thereof. In another aspect the one or more antigens comprise bacterial, fungal or viral antigens. In a specific aspect the antigen is a HA1 subunit of an influenza virus. In another aspect the one or more TLR2 ligands comprise heat-killed bacteria, lipoglycans, lipopolysaccharide, lipoteichoic acids, peptidoglycans, synthetic lipoproteins, zymosan or combinations and modifications thereof. In yet another aspect the composition comprises lipopolysaccharides comprising P. gingivalis LPS or E. coli LPS. In one aspect the composition increases secretion of IL-1β, IL-6, and IL-23 thereby leading to an enhanced Th-17 response. In another aspect the composition reduces Th2 cell responses.

### BRIEF DESCRIPTION OF THE DRAWINGS

For a more complete understanding of the features and advantages of the present invention, reference is now made to the detailed description of the invention along with the accompanying figures and in which:

FIGS. 1A-1F show antigen targeting to DCs via hDectin-1 resulting in HA1-specific CD4⁺ T cell responses: FIG. 1A reduced SDS-gel analysis of recombinant fusion proteins (Lane 1: anti-hDectin-1, Lane 2: anti-hDectin-1-HA1, and Lane 3: IgG4-HA1), FIG. 1B 293F cells transfected with full length of hDectin-1 and IFNDCs, FIG. 1C loading with different concentrations of anti-hDectin-1-HA1 or IgG4-HA1, and then stained with anti-human IgG-PE, FIG. 1D CFSE-labeled purified autologous CD4⁺ T cells were co-cultured with IFNDCs loaded with 10 or 1 μg/ml recombinant fusion proteins. Cell proliferation was measured on day 7. Three independent runs showed similar results, FIG. 1E CD4⁺ T cells restimulation after 7 days with 15 peptide pools (10 μM for each pool) for 4 h in the presence of Brefeldin A, and then stained with 7-AAD, anti-CD4, and anti-IFNγ antibodies (upper panels). Individual peptides (0.5 μM) in pool 8 were further tested (lower panels). Pep 32 from pool 2 was tested as a control, and FIG. 1F CD4⁺ T cells were restimulated with indicated peptides for 36 h, and then cytokines in culture supernatants were assessed. Error bars represent SD of triplicate assay. Two independent runs resulted in similar data;

FIG. 2 shows antigen targeting to DCs via hDectin-1 allows the detection of antigen-specific Th17 cells in healthy donors: Purified autologous CD4⁺ T cells were co-cultured IFNDCs loaded with 1 μg/ml anti-hDectin-1-HA1 for 7 days. CD4⁺ T cells were then restimulated with 0.5 μM peptides indicated for 36 h. Cytokines in the culture supernatants were measured. Peptide epitopes for seven healthy donors were determined by performing intracellular IFNγ staining with peptide pools and then individual peptides as described in FIG. 1E. Pep 18 and 32 were used as controls. Error bars represent SD of triplicate assay;

FIG. 3 shows a total 2×10⁵ CD4+ T cells co-cultured with 5×10³ IFNDCs targeted with 1 mg/ml anti-hDectin-1-HA1 for one week. Different concentrations of Pam3 was added into the co-culture of DCs and CD4+ T cells. CD4+ T cells were restimulated with indicated peptides (1 mM) for 48 h. Cytokines in culture supernatants were measured by Luminex;

FIGS. 4A-4D shows antigen targeting to DCs via hDectin-1 enhance antigen-specific Th17 cell responses by activating pre-existing antigen-specific Th17 memory cells. Purified autologous CD4⁺ T cells (CD45RA⁺CD45RO⁻ and CD45RA⁻CD45RO⁺) were co-cultured with IFNDCs loaded with 1 μg/ml anti-hDectin-1-HA1 for 7-8 days. CD4⁺ T cells were then restimulated with HA1-derived peptides for 36 h. Cytokines in culture supernatants were measured: FIG. 4A cells from four healthy donors were tested. Each line represents the data acquired with one donor, FIG. 4B data from three independent studies using cells from healthy donor. P values in FIGS. 4A and 4B were acquired by t-test, FIG. 4C IL-23 secreted by IFNDCs loaded with 1 μg/ml anti-hDectin-1-HA1, and FIG. 4D purified autologous total CD4⁺ T cells were co-cultured with IFNDCs loaded with 1 μg/ml anti-hDectin-1-HA1 for 7-8 days. CD4⁺ T cells were then restimulated with HA1-derived peptides for 36 h. Cytokines in culture supernatants were measured;

FIGS. 5A-5C show P. gingivalis LPS can promote antigen-specific Th17 cell responses elicited by IFNDCs targeted with anti-hDectin-1-HA1: FIG. 5A purified autologous CD4⁺ T cells were co-cultured IFNDCs loaded with 1 μg/ml anti-hDectin-1-HA1 for 7 days in the presence of 200 ng/ml P. gingivalis LPS (PG-LPS), 500 ng/ml poly I:C, 100 ng/ml E. coli LPS, or 200 ng/ml R848. CD4⁺ T cells were then restimulated with 0.5 μM peptides indicated for 36 h. Cytokines in the culture supernatants were measured, FIG. 5B different concentrations of P. gingivalis LPS were tested, and FIG. 5C 40 ng/ml PG-LPS were tested using cells from healthy donors;

FIGS. 6A and 6B show that Pam3 can promote antigen-specific Th17 cell responses elicited by IFNDCs targeted with anti-hDectin-1-HA1: FIG. 6A purified autologous CD4⁺ T cells were co-cultured IFNDCs loaded with 1 μg/ml anti-hDectin-1-HA1 for 7 days in the presence of different concentrations of Pam3. CD4⁺ T cells were then restimulated with 0.5 μM peptides indicated for 36 h. Cytokines in the culture supernatants were measured and FIG. 6B 40 ng/ml PG-LPS were tested using cells from healthy donors;

FIGS. 7A-7E show TLR2-mediated enhancement of antigen-specific memory Th17 cell responses are through IL-1β and is due to the activation of pre-existing memory Th17 cells, but not the induction of antigen-specific Th17 cells: FIG. 7A purified autologous CD4⁺ T cells (CD45RA⁺CD45RO⁻ and CD45RA⁻CD45RO⁺) were co-cultured IFNDCs loaded with 1 μg/ml anti-hDectin-1-HA1 for 7 days in the presence or absence of 40 ng/ml P. gingivalis LPS (PG-LPS). CD4⁺ T cells were then restimulated with 0.5 μM pep43 (donor #1), pep7 (donor #2), pep22 (donor #4), and pep22 (donor #5) for 36 h. Cytokines in the culture supernatants were measured. P values were acquired by t-test, FIG. 7B total CD4⁺ T cells were co-cultured with IFNDCs loaded with 1 ug/ml anti-hDectin-1-HA1 in the presence or absence 40 ng/ml PG-LPS for seven days. Cells were then stimulated with PMA and ionomycin, and stained for intracellular IFNγ and IL-17, FIG. 7C total RNA was extracted from CD4⁺ T cells in FIG. 7B. Relative expression levels of T-bet, Rorc, and GATA-3 were measured by RT-PCR. β-actin was used as a control. Three independent runs resulted in similar results and error bars are SD of the data from three runs, FIG. 7D 1×10⁵ IFNDCs loaded with 1 μg/ml anti-hDectin-1-HA1, 40 ng/ml P. gingivalis LPS or 1 μg/ml anti-hDectin-1-HA1 plus 40 ng/ml PG-LPS, and then incubated overnight. IL-10 and IL-6 levels in culture supernatants were measured, and FIG. 7E total CD4⁺ T cells were co-cultured with IFNDCs loaded with 1 ug/ml anti-hDectin-1-HA1 in the presence 40 ng/ml PG-LPS with indicated antibodies (10 g/ml of each) for seven days. CD4⁺ T cells were then restimulated with pep43 (donor #1), pep7 (donor #2), pep22 (donor #4), and pep22 (donor #5) for 36 h and IFNγ and IL-17 levels in the culture supernatants were measured. P values were acquired by t-test; and

FIGS. 8A and 8B show the phenotype of HA1-specific Th1 and Th17 CD4⁺ T cells elicited by DCs targeted with anti-hDectin-1-HA1: FIG. 8A purified autologous CD4⁺ T cells were co-cultured with IFNDCs loaded with 1 ug/ml anti-hDectin-1-HA1. Cells were restimulated with pep43 (donor #1) and stained for intracellular IFNγ and IL-17. Expression levels of CCR4, CCR5, CCR6, CCR9, CXCR3, integrin b7, and CD161 on both IFNγ⁺ and IL-17⁺HA1-specific CD4⁺ T cells were measured by flow cytometry, and FIG. 8B cells were restimulated with PMA/ionomycin and then stained for intracellular IFNγ and IL-17, and surface receptors.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Antigen Presenting Cells” (APC) refers to cells that are capable of activating T cells, and include, but are not limited to, certain macrophages, B cells and dendritic cells. “Dendritic cells” (DCs) refers to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein. Dendritic cell binding proteins refers to any protein for which receptors are expressed on a dendritic cell. Examples include GM-CSF, IL-1, TNF, IL-4, CD40L, CTLA4, CD28, and FLT-3 ligand.

The term “vaccine composition” as used in the present invention is intended to indicate a composition which can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes or both. As used herein the term “antigen” refers to any antigen which can be used in a vaccine, whether it involves a whole microorganism or a subunit, and whatever its nature: peptide, protein, glycoprotein, polysaccharide, glycolipid, lipopeptide, etc. They may be viral antigens, bacterial antigens or the like; the term “antigen” also comprises the polynucleotides, the sequences of which are chosen so as to encode the antigens whose expression by the individuals to which the polynucleotides are administered is desired, in the case of the immunization technique referred to as DNA immunization. They may also be a set of antigens, in particular in the case of a multivalent vaccine composition which comprises antigens capable of protecting against several diseases, and which is then generally referred to as a vaccine combination or in the case of a composition which comprises several different antigens in order to protect against a single disease, as is the case for certain vaccines against whooping cough or the flu, for example. The term “antibodies” refers to immunoglobulins, whether natural or partially or wholly produced artificially, e.g. recombinant. An antibody may be monoclonal or polyclonal. The antibody may, in some cases, be a member of one or a combination immunoglobulin classes, including: IgG, IgM, IgA, IgD, and IgE.

The term “adjuvant” refers to a substance that enhances, augments or potentiates the host's immune response to a vaccine antigen.

The term “gene” is used to refer to a functional protein, polypeptide or peptide-encoding unit. As will be understood by those in the art, this functional term includes both genomic sequences, cDNA sequences or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA) or analogs of naturally-occurring nucleotides (e.g., α-enantiomeric forms of naturally-occurring nucleotides) or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used in this application, the term “amino acid” refers to the one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “in vivo” refers to being inside the body. The term “in vitro” used as used in the present application is to be understood as indicating an operation carried out in a non-living system.

As used herein, the term “treatment” or “treating” includes any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The instant invention describes methods and compositions for enhancing Th17 cell responses by targeting antigens to human dendritic cells (DCs) via Dectin-1.

IL-17-producing T cells (Th17 cells) are crucial components of protective immunity against bacterial, fungal, and viral infections. Thus, the enhancement of pathogen-specific Th17 cells in memory pools is of importance for protection against subsequent infections. However, pathogen-specific human memory Th17 cells have been poorly understood because of their low frequencies in healthy individuals. Dectin-1, a c-type lectin-like pattern-recognition receptor, has been associated with Th17 cell responses during bacterial and fungal infections. The present invention demonstrates that healthy individuals maintain broad ranges of pathogen (Influenza viruses)-specific Th17 cells. This was achieved by targeting antigens (HA1 subunit, A/PR8/34) to human dendritic cells (DCs) via Dectin-1 using recombinant proteins of agonistic anti-hDectin-1 fused to HA1 (anti-hDectin-1-HA1). HA1-specific Th17 cell responses elicited with anti-hDectin-1-HA1 was further enhanced by P. gingivalis lipopolyssaccharide (LPS) and Pam3, but not poly I:C, E. coli LPS, or 8848. The TLR2 ligand-mediated enhancement of Th17 cell responses were mainly dependent on IL-1b secreted by DCs. The findings of the present invention demonstrate that HA1-specific Th17 cell responses elicited by anti-hDectin-1-HA1 alone or anti-hDectin-1-HA1 plus TLR2 were not the results of priming naïve CD4+ T cells, but the results of activation of pre-existing HA1-specific memory Th17 cells.

IL-17-producing Th17 CD4+ T cells (Th17 cells) has been broadly linked to the pathogenesis of multiple autoimmune diseases (1-3). However, recent compelling evidence indicates that Th17 cells are crucial for protective immunity against many mucosal and systemic infections of bacteria (5-7)(4), fungi (8-11), viruses (12-14), and parasites (15). Th17 cells also play an important role in vaccine-induced protective immunity against infections (6, 12, 13, 16-18). Thus, understanding the pathways for the enhancement of pathogen-specific Th17 cells is important to mount potent protective immunity against such infections. Early activation and expansion of pre-existing pathogen-specific Th17 cells in memory pools are also thought to be an efficient way to mount protective immunity against subsequent infections by the same pathogens or pathogens sharing antigenic epitopes.

The induction of mouse Th17 cells from naive T cells is initially dependent on the presence of TGF-β, IL-21, and IL-6, and at later stages on IL-23 (19). In humans, the differentiation of naïve T cells into Th17 cells is associated with IL-1, IL-6 (20, 21) and TGF-β (22-24). In addition, IL-23 and IL-113 induce the production of IL-17 from human memory CD4+ T cells (25, 26). However, many of these studies have been conducted in limited experimental conditions, such as using APC-free cultures with anti-CD3/CD28 stimuli, addition of exogenous cytokines, and neutralization of IFN-γ/IL-4. In addition, Th17 cell responses elicited by polyclonal stimuli or by activating T cells via allogeneic recognitions may not always represent the pathogen-specific Th17 cell responses elicited during and after infections. Furthermore, it is still not clear that memory Th17 cells specific for pathogen-derived peptide-MHC class II exist as discrete Th17 subset in vivo because such cells are difficult to detect in normal hosts.

In the present invention the inventors tested the presence of pathogen (Influenza viruses)-specific Th17 cells in healthy donors by targeting antigens (HA1 subunit) to DCs via human Dectin-1 (hDectin-1). Antigen targeting to DCs is an efficient way to elicit antigen-specific T cell responses (28, 29). The inventors demonstrate that healthy individuals maintain broad ranges of HA1-specific memory Th17 cells that could be greatly enhanced by TLR2 ligands. The findings of the present invention also indicate that TLR2 ligand-mediated enhancement of HA1-specific Th17 responses was the results of activation of pre-existing memory Th17 cells.

Cells: Peripheral blood mononuclear cells (PBMCs) of healthy volunteers were fractionated by elutriation, according to Institutional Review Board guidelines. IL-4DCs and IFNDCs were generated by culturing monocytes from healthy donor in serum free media (Cellgenix, Germany) supplemented with GM-CSF (100 ng/ml) and IFNa α (500 U/ml) (IFNDCs) or GM-CSF (100 ng/ml) and IL-4 (50 ng/ml). The medium was replenished with cytokines on day 1 for IFNDCs and on day 3 for IL-4DCs. IFNα, IL-4 and GM-CSF were from the Pharmacy in Baylor University Medical Center (Dallas, Tex.). Autologous CD4+ T cells were purified using EasySep Human CD4+ T Cell Enrichment Kit (Stemcell, CA). Monocytes and B cells from PBMCs were purified with EasySep Human CD4⁺ T Cell Enrichment Kit (StemCell, CA). Naïve (CD45RA+CD45RO−) and memory CD4+ T cells (CD45RA−CD45RO+) (purity>99.2%) were purified by FACS Aria (BD Biosciences).

Antibodies and reagents: Anti-CD4, anti-IFNγ, anti-CCR6, and anti-CXCR3 were purchased from Biolegend (CA). Anti-CCR4, anti-CCR5, anti-CCR9, anti-IL-IR1, and anti-CCR7 were from R&D Systems (MN). Anti-437 integrin, anti-CD161, anti-CD45RA, and anti-CD45RO were purchased from BD Biosciences (CA). Anti-IL-17 (eBioscience, CA) and anti-human IgG (Jackson ImmunoResearch Laboratories, PA) were used. Neutralizing anti-IL-23p19 and control IgG were purchased from R&D Systems (CA). GolgiPlug was purchased from BD Pharmingen (CA). CFSE (Molecular probes, Oregon) was used for measuring CD4⁺ T cell proliferation. LPS from P. gingivalis, LPS from E. coli, Pam3CSK4, poly I:C, and R848 were purchased from Invivogen (OR).

Peptides: Overlapping (staggered by 11 amino acids) 17-mer peptides spanning the entire HA1 subunit of HA (A/PR/8/34 H1N1) were synthesized by Biosynthesis (TX).

DCs and CD4+ T cell co-cultures: 1-2×10⁵ CFSE-labeled purified CD4+ T cells were co-cultured with 5×10³ DCs in complete RPMI 1640 (GIBCO, NY) supplemented with 25 mM HEPES buffer, 2 mM L-glutamine, 1% nonessential amino-acids, 1 mM sodium pyruvate, 50 units/ml penicillin, 50 μg/ml streptomycin, and 10% AB serum (GemCell, CA). DCs were loaded with recombinant fusion proteins indicated for at least 6 h before mixing with the CD4+ T cells. After 7 days, CD4+ T cell proliferation was tested by measuring CFSE-dilution. In some studies, anti-IL-23p19, anti-IL-6 and anti-IL-6R, anti-IL-1b or control IgG (10 mg/ml) was added into the co-cultures of DCs and CD4+ T cells.

Assessment of antigen-specific CD4+ T cell responses: CD4+ T cells were restimulated with indicated HA1-derived peptides (2 mM) for 4 h in the presence of Brefeldin A, and then stained with 7-AAD, anti-CD4 and anti-IFNg antibodies labeled with fluorescent dyes. CD4+ T cells expressing IFNg were detected by flow cytometry. CD4+ T cells were also stained for both IL-17 and IFNg during restimulation with 50 ng/ml phorbol 12-myristate 13-acetate (PMA) and 1 mg/ml ionomycin. In separate experiments, CD4+ T cells were stimulated with indicated peptides for 36 h, and then culture supernatants were harvested for measuring cytokines and chemokines. Cytokine multiplex analysis was carried out using the Beads cytokine assay kit (seromap) as per the manufacturer's protocol. Cytokine concentrations were measured with a Bio-Plex Luminex instrument (Biorad, CA). To measure IL-23 secreted from DCs loaded with recombinant fusion proteins, 1×10⁵ DCs were loaded with 1 mg/ml anti-hDectin-1-HA1 or IgG4-HA1. After 24 h, IL-23 in culture supernatants was measured using human IL-23 ELISA KIT (eBiosciences).

Expression and purification of chimeric recombinant mAbs fused to HA1: Total RNA was prepared from hybridoma cells using RNeasy kit (Qiagen, CA) and used for cDNA synthesis and PCR (SMART RACE kit, BD Biosciences). PCR products were then cloned (pCR2.1 TA kit, Invitrogen) and characterized by DNA sequencing (MC Lab, CA). Using the derived sequences for the mouse heavy (H) and light (L) chain variable (V)-region cDNAs, specific primers were used to PCR amplify the signal peptide and V-regions while incorporating flanking restriction sites for cloning into expression vectors encoding downstream human IgG4H regions. The vector for expression of chimeric mVκ-hIgκ was built by amplifying residues 401-731 (gi|63101937|) flanked by Xho I and Not I sites and inserting this into the Xho I-Not I interval of pIRES2-DsRed2 (BD Biosciences). PCR was used to amplify the mAb Vk region from the initiator codon, appending a Nhe I or Spe I site then CACC, to the region encoding (e.g., residue 126 of gi|76779294|), appending a distal Xho I site. The PCR fragment was then cloned into the Nhe I-Not I interval of the above vector. The control human IgGκ sequence corresponds to Gi|49257887| residues 26-85 and gi|21669402| residues 67-709. The control human IgG4H vector corresponds to residues 12-1473 of gi|19684072| with S229P and L236E substitutions, which stabilize a disulphide bond and abrogate residual FcR interaction (30), inserted between the Bgl II and Not I sites of pIRES2-DsRed2 while adding the sequence 5′gctagctgattaattaa 3′ (SEQ ID NO: 7) instead of the stop codon. PCR was used to amplify the mAb VH region from the initiator codon, appending CACC then a Bgl II site, to the region encoding residue 473 of gi|19684072|. The PCR fragment was then cloned into the Bgl II-Apa I interval of the above vector.

The Flu HA1 antigen coding sequence is a CipA protein [Clostridium thermocellum] gi|479126| residues 147-160 preceding hemagglutinin [Influenza A virus (A/Puerto Rico/8/34(H1N1))] gi|126599271| residues 18-331 with a P321L change and with 6 C-terminal His residues was inserted between the H chain vector Nhe I and Not I sites to encode recombinant antibody-HA1 fusion proteins. Stable CHO-S cells were grown in GlutaMAX and HT media (Invitrogen) and recombinant proteins were purified by protein A column chromatography. Purified proteins were confirmed by reduced-SDS gel analysis.

Binding of recombinant fusion proteins to hDectin-1 and APCs: 2×10⁵ cells (293F cells transfected with full length of hDectin-1 and IFNDCs) were incubated with different concentrations of recombinant fusion proteins (anti-hDectin-1-HA1 and IgG4-HA1) for 20 min at 4° C. Cells were then washed twice with 2% FCS in PBS, and then stained with secondary antibody, anti-human IgG-PE, for 20 min. Cells were analyzed by flow cytometry.

RT-PCR: Total RNA was isolated from cell lysates using QIAGEN RNeasy “Mini” spin columns according to the instructions of the manufacturer and then subjected to a 20 mL cDNA synthesis reaction (Promega). Random primers were used as primer. 2.5 mL cDNA was used for PCR amplification. The primer sequences and PCR temperature profiles for T-bet, RORC, GATA-3, and b-actin is provided in Table 1. A total of 4 μL, of the reverse transcriptase (RT)-PCR reactions was electrophoresed through a 4-12% Bis-Tris gel and stained with ethidium bromide for visualization under ultraviolet light.

TABLE 1
Primer sequences and PCR temperature profiles.

| | | PCR Temperature |
|---|---|---|
| Primer | Sequence | Profile |
| T-bet | T-bet forward: CACTACAGGATGTTTGTGGACGTG | 5 minutes of |
| | (SEQ ID NO: 1) | pretreatment at |
| | T-bet reverse: CCCCTTGTTGTTTGTGAGCTTTAG | 94° C. |
| | (SEQ ID NO: 2) | |
| RORC | RORc forward: TCTGGAGCTGGCCTTTCATCATCA | 30 cycles at 94° C. |
| | (SEQ ID NO: 3) | for 15 seconds |
| | RORc reverse: TCTGCTCACTTCCAAAGAGCTGGT | |
| | (SEQ ID NO: 4) | |
| B-actin | ACTB forward: GGATGCAGAAGGAGATCACT | 72° C. for 1 minute |
| | (SEQ ID NO: 5) | |
| | ACTB reverse: CGATCCACACGGAGTACTTG | |
| | (SEQ ID NO: 6) | |

Statistical Analysis: Statistical significance was determined using the Student's t-test and significance was set at P<0.05. Spearman's correlations statistics were used.

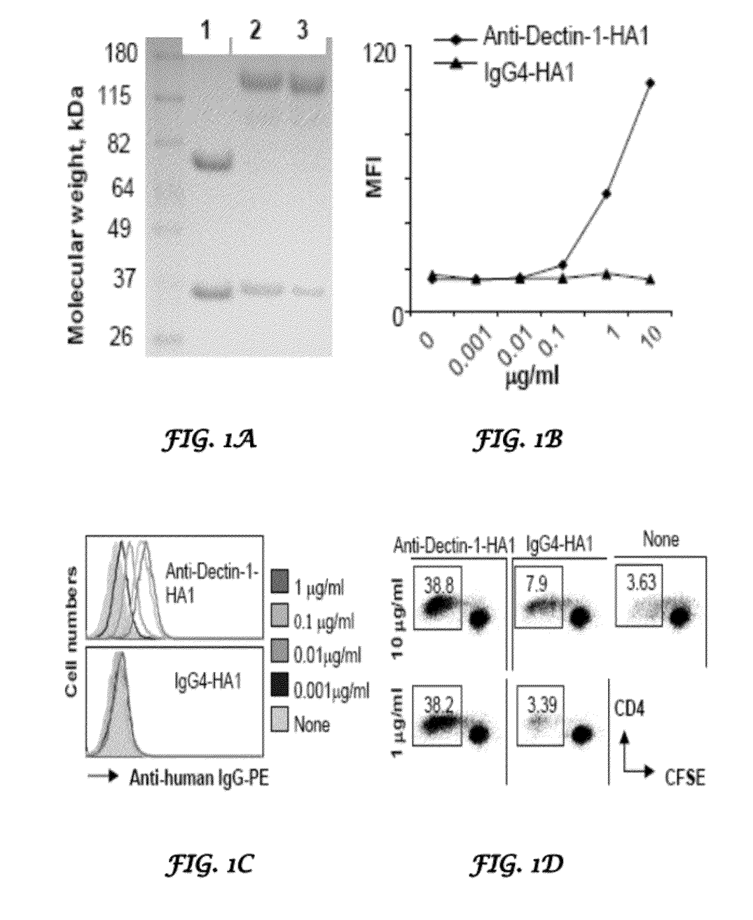

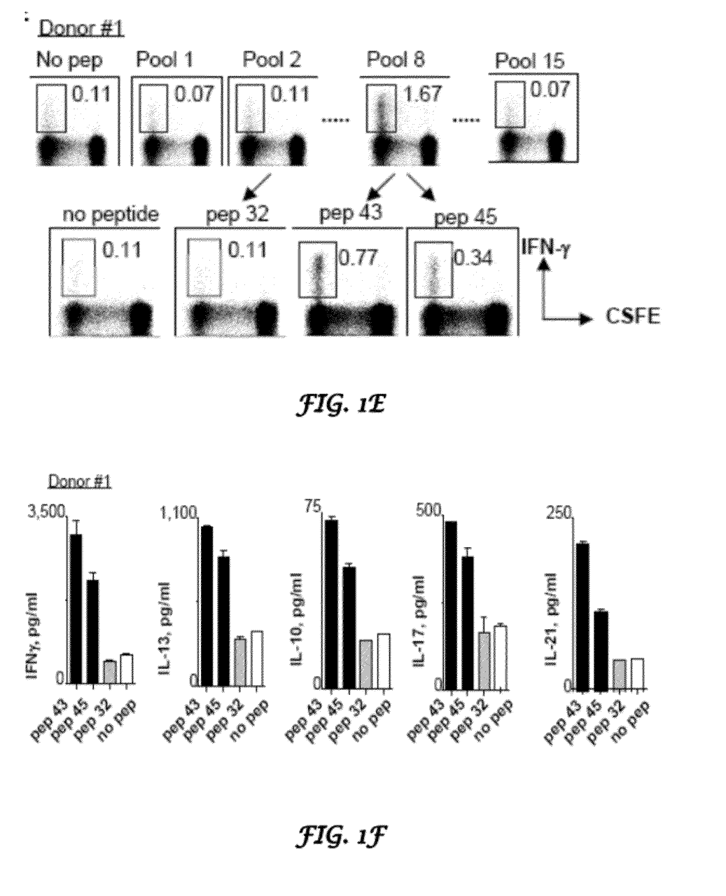

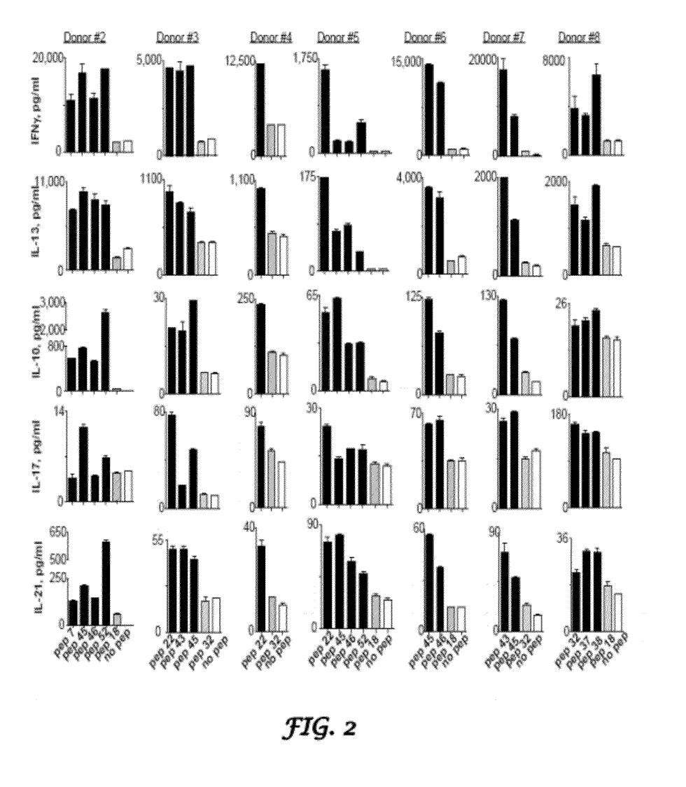

Anti-hDectin-1-HA1 can target hDectin-1 molecules expressed on DCs: To target HA1 to DCs via hDectin-1, recombinant proteins of an agonistic anti-hDectin-1 mAb (Ni et al. 2010) fused to HA1 subunit of influenza viral hemagglutinin (A/PR8/34, H1N1) (anti-hDectin-1-HA1) were generated and analyzed in reduced SDS-gel (FIG. 1A). A human IgG₄-HA1 fusion protein was made as a control. Anti-hDectin-1 mAb was engineered as a chimera containing mouse V-region and human IgG4 Fc with two site mutations to abrogate residual non-specific binding capacity to Fc receptors (30).

Binding capacity of the two recombinant fusion proteins to hDectin-1 molecules were assessed. Anti-hDectin-1-HA1, but not IgG4-HA1, bound efficiently to 293F cells transfected with full length of hDectin-1 molecules in a concentration dependent manner (FIG. 1B). Similarly, anti-hDectin-1-HA1 bound to IFNDCs more efficiently than IgG4-HA1 (FIG. 1C), suggesting that anti-hDectin-1-HA1 target hDectin-1 molecules expressed on DCs. In addition, IFNDCs loaded with anti-hDectin-1-HA1 induced greater proliferation of the purified autologous CD4+ T cells than IgG4-HA1 did (FIG. 1D). IFNDCs loaded with either 10 or 1 mg/ml anti-hDectin-1-HA1 induced similar levels of CD4+ T cell proliferation (>38%). In contrast, 10 mg/ml IgG4-HA 1 induced only 7.9% of CD4+ T cell proliferation. 1 mg/ml IgG4-HA1 induced background levels of CD4+ T cell proliferation. Thus, it can be concluded that anti-hDectin-1-HA1 can target hDectin-1 molecules expressed on DCs, and this resulted in the enhanced proliferation of autologous CD4+ T cells.

TABLE 2
Predicted binding scores of individual peptide to corresponding MHC class II in
each donor tested in this study.

| | | Pep- | Amino acid | | Binding scores | |
|---|---|---|---|---|---|---|
| Donors | HLA types | tides | residues | Peptide sequences | (ABR score) | |
| Donor #1 | HLA-DRB 1*03 | pep 7 | 37-53 | LEKNVTVTHSVNLLEDS | 603.2 | SEQ ID NO: 8 |
| | | pep 45 | 262-278 | GNLIAPWYAFALSRGFG | 1000000 | SEQ ID NO: 9 |
| | | pep 46 | 268-284 | WYAFALSRGFGSGIITS | 1000000 | SEQ ID NO: 10 |
| | | pep 52 | 304-320 | SSLPFQNVHPVTIGECP | 1000000 | SEQ ID NO: 11 |
| | HLA-DRB1*07 | pep 7 | 37-53 | LEKNVTVTHSVNLLEDS | 709.8 | SEQ.ID NO: 8 |
| | | pep 45 | 262-278 | GNLIAPWYAFALSRGFG | 1000000 | SEQ ID NO: 9 |
| | | pep 46 | 268-284 | WYAFALSRGFGSGIITS | 95088.6 | SEQ ID NO: 10 |
| | | pep 52 | 304-320 | SSLPFQNVHPVTIGECP | 192.5 | SEQ ID NO: 11 |
| | HLA-DQB 1*02 | | NA | | NA | |
| Donor #2 | HLA-DRB1*01 | pep 43 | 250-266 | LEPGDTIIFEANGNLIA | 669 | SEQ ID NO: 12 |
| | | pep 45 | 262-278 | GNLIAPWYAFALSRGFG | 12.4 | SEQ ID NO: 9 |
| | HLA-DQB1*05 | | NA | | NA | |
| Donor #3 | HLA-DRB1*13 | pep 22 | 126-142 | SSFERFEIFPKESSWPN | 1000000 | SEQ ID NO: 13 |
| | | pep 43 | 250-266 | LEPGDTIIFEANGNLIA | 1000000 | SEQ ID NO: 12 |
| | | pep 45 | 262-278 | GNLIAPWYAFALSRGFG | 83821.7 | SEQ ID NO: 9 |
| | HLA-DRB1*15 | pep 22 | 126-142 | SSFERFEIFPKESSWPN | 461455.1 | SEQ ID NO: 13 |
| | | pep 43 | 250-266 | LEPGDTIIFEANGNLIA | 1000000 | SEQ ID NO: 12 |
| | | pep 45 | 262-278 | GNLIAPWYAFALSRGFG | 12070.6 | SEQ ID NO: 9 |
| | HLA-DRB3/4/5*03 | | NA | | NA | |
| | HLA-DQB1*06 | | NA | | NA | |
| Donor #4 | HLA-DRB1*03 | pep 22 | 126-142 | SSFERFEIFPKESSWPN | 1000000 | SEQ ID NO: 13 |
| | HLA-DRB1*11 | pep 22 | 126-142 | SSFERFEIFPKESSWPN | 18064.8 | SEQ ID NO: 13 |
| | HLA-DQB1*03 | | NA | | NA | |
| Donor #5 | HLA-DRB1*07 | pep 22 | 126-142 | SSFERFEIFPKESSWPN | 5796.6 | SEQ ID NO: 13 |
| | | pep 45 | 262-278 | GNLIAPWYAFALSRGFG | 95088.6 | SEQ ID NO: 9 |
| | | pep 46 | 268-284 | WYAFALSRGFGSGIITS | 95088.6 | SEQ ID NO: 10 |
| | | pep 52 | 304-320 | SSLPFQNVHPVTIGECP | 192.5 | SEQ ID NO: 11 |
| | HLA-DRB1*11 | pep 22 | 126-142 | SSFERFEIFPKESSWPN | 18064.8 | SEQ ID NO: 13 |
| | | pep 45 | 262-278 | GNLIAPWYAFALSRGFG | 81720.8 | SEQ ID NO: 9 |
| | | pep 46 | 268-284 | WYAFALSRGFGSGIITS | 81720.8 | SEQ ID NO: 10 |
| | | pep 52 | 304-320 | SSLPFQNVHPVTIGECP | 18059.9 | SEQ ID NO: 11 |
| | HLA-DQB1*02 | | NA | | NA | |
| | HLA-DQB1*03 | | NA | | NA | |
| Donor #6 | HLA-DRB1*07 | pep 45 | 262-278 | GNLIAPWYAFALSRGFG | 95088.6 | SEQ ID NO: 9 |
| | | pep 46 | 268-284 | WYAFALSRGFGSGIITS | 95088.6 | SEQ ID NO: 10 |
| | HLA-DRB1*13 | pep 45 | 262-278 | GNLIAPWYAFALSRGFG | 83821.7 | SEQ ID NO: 9 |
| | | pep 46 | 268-284 | WYAFALSRGFGSGIITS | 101038.1 | SEQ ID NO: 10 |
| | HLA-DQB1*02 | | NA | | NA | |
| | HLA-DQB1*06 | | NA | | NA | |
| Donor #7 | HLA-DRB1*07 | pep 43 | 250-266 | LEPGDTIIFEANGNLIA | 1000000 | SEQ ID NO: 12 |
| | | pep 45 | 262-278 | GNLIAPWYAFALSRGFG | 95088.6 | SEQ ID NO: 9 |
| | HLA-DRB1*08 | pep 43 | 250-266 | LEPGDTIIFEANGNLIA | 1000000 | SEQ ID NO: 12 |
| | | pep 45 | 262-278 | GNLIAPWYAFALSRGFG | 1233.33 | SEQ ID NO: 9 |
| | HLA-DQB1*02 | | NA | | NA | |
| | HLA-DQB1*04 | | NA | | NA | |
| Donor #8 | HLA-DRB1*07 | pep 32 | 186-202 | EKEVLVLWGVHHPPNIG | 2264.27 | SEQ ID NO: 14 |
| | | pep 37 | 215-231 | VSVVSSHYSRRFTPEIA | 344.05 | SEQ ID NO: 15 |
| | | pep 38 | 221-237 | HYSRRFTPEIAKRPKVR | 4060.99 | SEQ ID NO: 16 |
| | HLA-DRB1*08 | pep 32 | 186-202 | EKEVLVLWGVHHPPNIG | 2799.61 | SEQ ID NO: 14 |
| | | pep 37 | 215-231 | VSVVSSHYSRRFTPEIA | 1346.63 | SEQ ID NO: 15 |
| | | pep 38 | 221-237 | HYSRRFTPEIAKRPKVR | 14251.05 | SEQ ID NO: 16 |
| | HLA-DQB1*02 | | NA | | NA | |
| | HLA-DQB1*04 | | NA | | NA | |

HA1 targeted to DCs via hDectin-1 activate different types of HA1-specific CD4+ T cells: Antigen specificity of the proliferating CD4+ T cells (FIG. 1D) was tested by measuring intracellular IFNg expression. Fifteen clusters of HA1-derived peptides (11-12 peptides in one cluster, 17-mers overlapping by 11 amino acids) were first screened (upper panels in FIG. 1E), and then individual peptides in the positive cluster (cluster 8) were further tested (lower panels in FIG. 1E). Significant numbers of CFSE-CD4+ T cells expressed intracellular IFNg during restimulation with 1 mM peptides 43 and 45. Pep32 from pool 2 was tested as a negative control. The inventors then subsequently measured the amount of cytokines (IFNg, IL-13, IL-10, IL-17, and IL-21) secreted from the CD4+ T cells stimulated with HA1-derived pep43, pep45, and pep32 (FIG. 1F). Both pep43 and pep45 induced CD4+ T cells to secrete significant amounts of the cytokines tested. This suggests that HA1 targeted to DCs via hDectin-1 can elicit HA1-specific CD4+ T cell responses. Although monocyte-derived DCs, B cells, and monocytes in peripheral blood mononuclear cells (PBMC) express similar levels of hDectin-1, DCs were far more efficient than other APCs for inducing CD4+ T cell proliferation as well as activating HA1-specific CD4+ T cells (data not shown). Influenza viral infections induce IFNa secretion from immune cells, including DCs (31), and IFNa can induce monocyte differentiation into DCs (32). IFNDCs generated in the presence of IFNa and GM-CSF were more potent than IL-4DCs generated with IL-4 and GM-CSF for proliferation and activation of HA1-specific CD4+ T cells (data not shown).

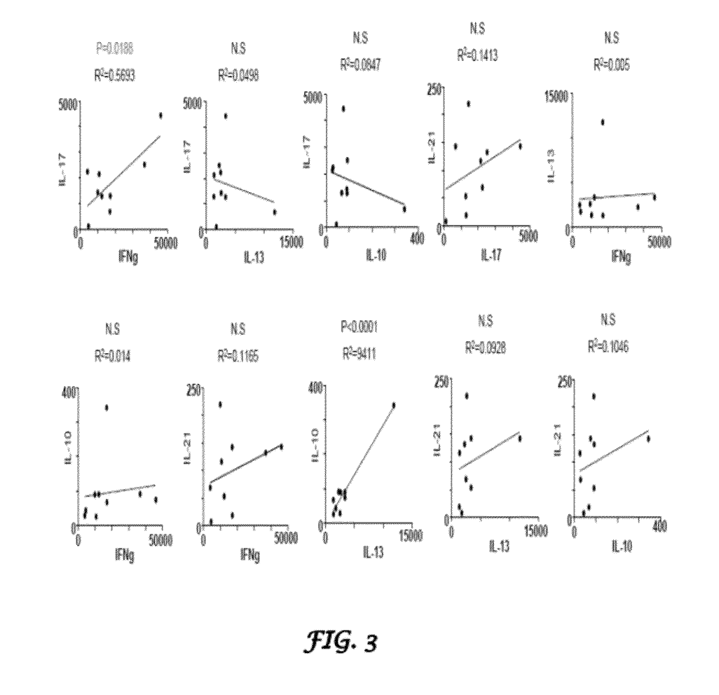

To extend the findings described in FIG. 1F, the inventors assessed the types of HA1-specific CD4+ T cells present in 7 healthy individuals (FIG. 2). First of all, all healthy individuals maintained significant levels of HA1-specific CD4+ T cells, including IL-17-producing cells. The magnitudes, as measured by the levels of cytokines secreted, of different types of HA1-specific CD4+ T cells were highly variable among peptide epitopes as well as among individuals. For example, all IFNg-inducing peptides (pep7, pep45, pep46, and pep52) in donor #2 also induced CD4+ T cells to secrete significant amounts of IL-13. However, pep52-specific CD4+ T cells produced higher amounts of both IL-10 and IL-21 than CD4+ T cells specific for pep7, 45, and pep46. In addition, the magnitudes of HA1-derived peptide specific Th17 responses were not correlated to the magnitudes of other types of CD4+ T cell responses that were specific for the same HA1-derived peptides (FIG. 3). As an example, HA 1-specific CD4+ T cells in donor #2 secreted greater amounts of IFNg, IL-13, IL-10, and IL-21 than did HA1-specific CD4+ T cells in donor #3, but CD4+ T cells in donor #3 secreted greater amount of IL-17 than did CD4+ T cells in donor #2. Detailed information for HLA types of healthy donors tested and predicted binding scores of individual peptides to corresponding class II types are summarized in Table 2.

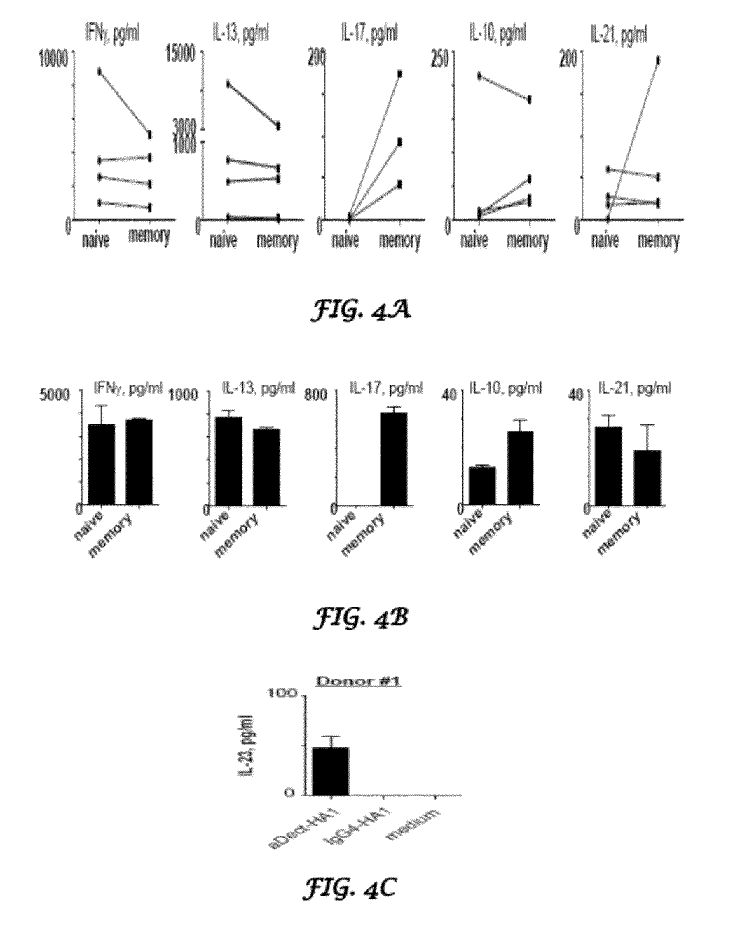

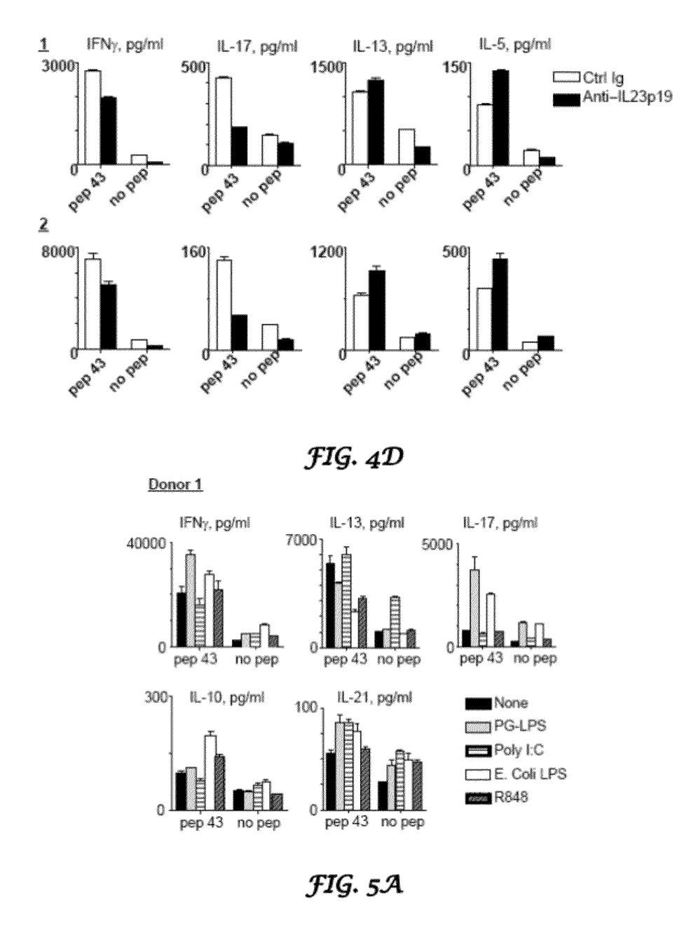

Based on the data in FIGS. 1F and 2, it can be assumed that healthy individuals maintain pathogen-specific memory Th17 cells. The inventors then tested if the types of HA 1-specific CD4+ T cell responses observed were the results of the activation of pre-existing HA 1-specific memory T cells. Two populations of CD4+ T cells (CD45RA+CD45RO− and CD45RA-CD45RO+) were separately tested. The possibility that the responses observed with CD45RA+CD45RO− CD4+ T cell population might also be the results of the activation of contaminated HA1-specific memory T cells was not eliminated. However, the inventors assumed that the responses observed with CD45RA−CD45RO+ T cells are mainly due to the activation of memory T cells. FIG. 4A shows that both populations of CD4+ T cells (CD45RA+CD45RO− and CD45RA−CD45RO+) resulted in similar levels of IFNg-, IL-13-, IL-10, and IL-21-producing HA1-specific responses. In contrast, significant levels of HA1-specific Th17 responses were observed only from CD45RA−CD45RO+CD4+ T cells. This suggestes that HA1-specific Th17 cell responses observed in healthy donors were mainly due to the activation of pre-existing HA1-specific Th17 memory cells. FIG. 4B presents the data from three independent studies using cells from the same donor.

Anti-hDectin-1-HA1 could activate DCs to secrete IL-23 (FIG. 4C) that can contribute to the enhanced Th17 and Th1, and reduced Th2 cell responses (FIG. 4D). However, it was important to note that the magnitudes of IL-17 cell responses observed in FIG. 2 were not correlated with the amounts of IL-23 secreted by DCs from the same donors (data not shown). For example, DCs from donor #2 and #5 secreted higher levels of IL-23 (≈80 pg/ml) than DCs from donor #1, but HA1-specific CD4+ T cells in donor #1 secreted greater amount of IL-17 than CD4+ T cells in donor #2 or #5. Taken together, the data demonstrates that DCs targeted with ant-hDectin-1-HA1 could enhance HA1-specific Th17 cell responses by activating pre-existing memory Th17 cells.

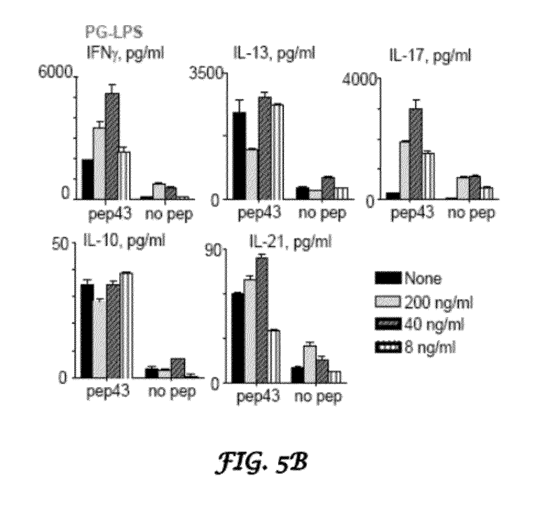

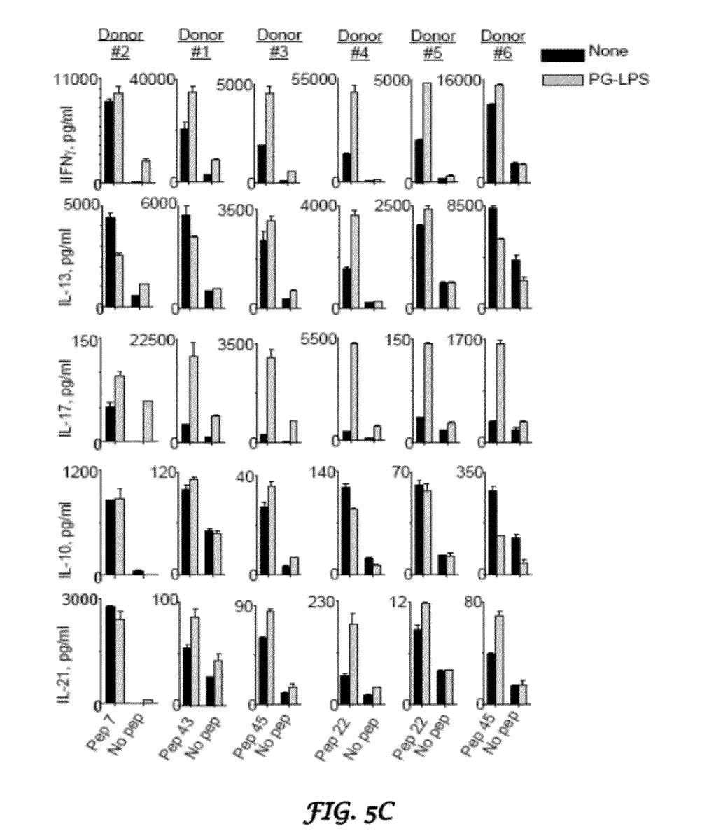

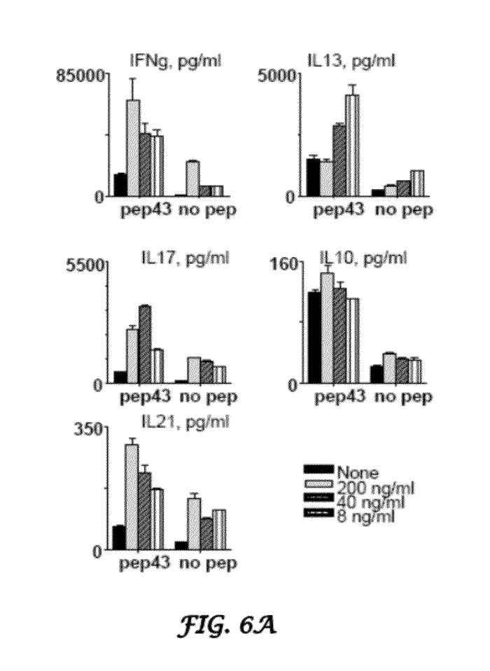

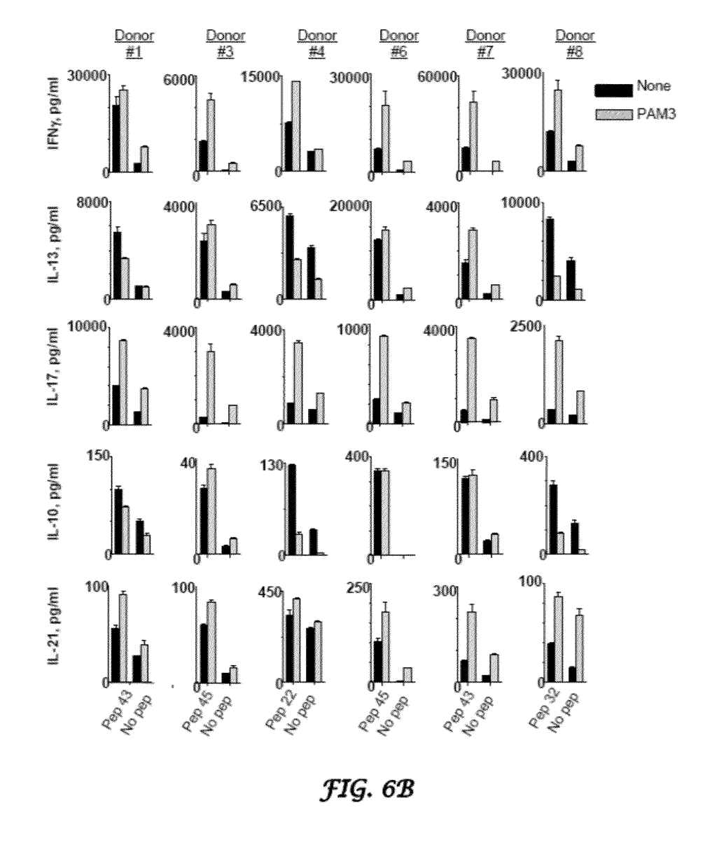

TLR2 ligands can promote the enhancement of HA1-specific memory Th17 cell responses: The inventors tested whether TLR ligands could further enhance the HA1-specific Th17 cell responses elicited by DCs targeted with anti-hDectin-1-HA1 (FIG. 5A). Only P. gingivalis LPS and E. coli LPS significantly enhanced HA1-specific Th17 cell responses. Neither poly I:C nor R848 (TLR7/8 ligand) enhanced Th17 cell responses. Although E. coli LPS enhanced Th17 cell responses, it also promoted IL-10-producing HA1-specific CD4+ T cell responses. P. gingivalis LPS was further titrated using cells from donor #1 (FIG. 5B). Both Th1 and Th17 responses peaked at 40 ng/ml P. gingivalis LPS. 40 ng/ml P. gingivalis LPS also enhanced HA1-specific Th21 CD4+ T cell responses. Interestingly, P. gingivalis LPS, at high dose (200 ng/ml), resulted in decreased HA1-specific Th2 type CD4+ T cell responses. It was also important to note that a low dose of P. gingivalis LPS (8 ng/ml) could enhance Th17 responses, but not Th1 responses. The inventors then extended the studies by testing cells from other healthy donors tested in FIG. 2. FIG. 5C shows that P. gingivalis LPS resulted in enhanced HA 1-specific Th17 cell responses in all 6 donors. P. gingivalis LPS also promoted both Th1 and Th21 CD4+ T cell responses in donors tested except for donor #2. Both IL-13 and IL-10-producing CD4+ T cell responses were variable among donors. Data in FIG. 3 show that P. gingivalis LPS enhanced the correlations between Th17 and Th1 responses as well as Th17 and Th21 responses to the same peptide epitopes tested. In addition to P. gingivalis LPS, the inventors tested another TLR2 ligand, Pam3 (FIGS. 6A and 6B). Both P. gingivalis LPS and Pam 3 resulted in enhanced HA1-specific Th17 cell responses. P. gingivalis LPS can bind to TLR2 (36, 37).

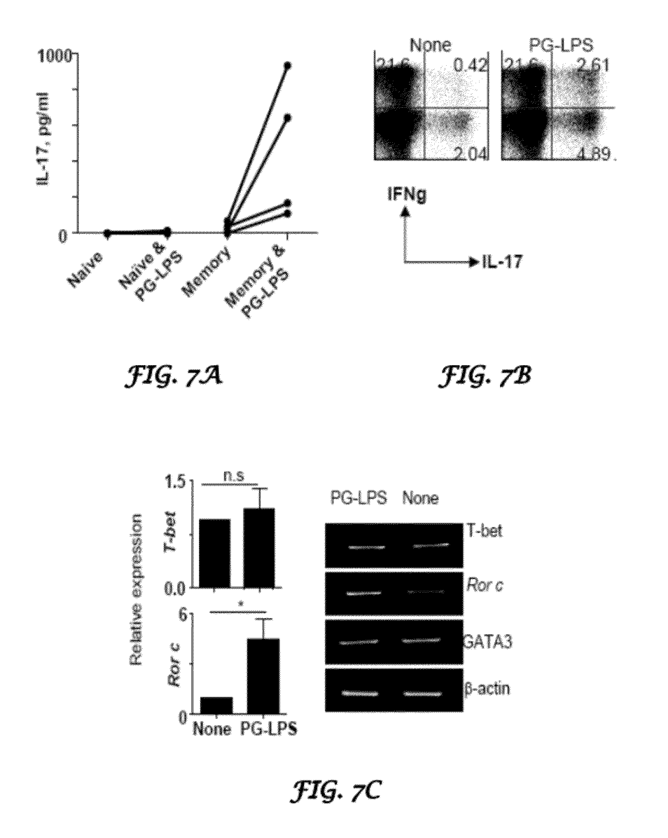

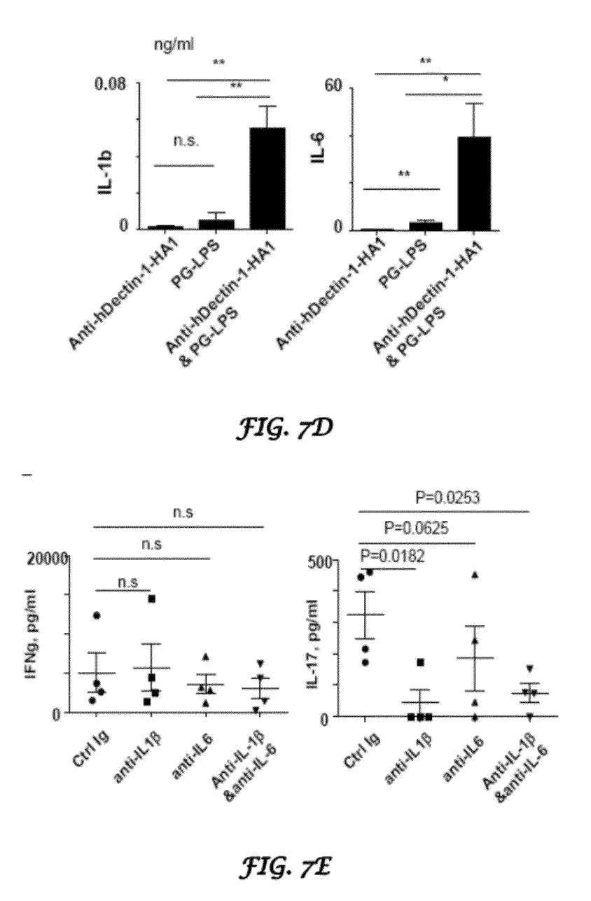

TLR2 ligands promote antigen-specific memory Th17 cell responses by inducing DCs to produce IL-1b: To test if the TLR2 ligands-mediated enhancement of HA1-specific Th17 cell responses were due to the activation of pre-existing memory Th17 cells, purified CD45RA+CD45RO− and CD45RA−CD45RO+ populations were tested (FIG. 7A). P. gingivalis LPS significantly enhanced HA1-specific Th17 cell responses in the studies using CD45RA+CD45RO−, but not CD45RA−CD45RO+ population. This suggested that the TLR2 ligands-mediated HA1-specific Th17 cell responses were mainly due to the activation of pre-existing memory Th17 cells. FIG. 7B shows that memory Th17 cells activated in the presence of P. gingivalis LPS express either IL-17 alone or IL-17 and IFNg. Pam3 also resulted in a similar response (data not shown). Consistently, T cells cultured in the presence of TLR2 ligands showed a significant increase in the expression of Rorc (FIG. 7C).

DCs loaded with anti-hDectin-1-HA1 plus TLR2 ligands secreted greater amounts of IL-1b and IL-6 than DCs loaded with either anti-hDectin-1-HA1 or P. gingivalis LPS alone (FIG. 7D). Thus, the inventors tested whether IL-1b or IL-6 could contribute to the TLR2 ligand-mediated enhancement of HA1-specific memory Th17 cell responses. Blocking IL-1b in the co-culture of DCs and CD4+ T cells resulted in decreased levels of IL-17 production from T cells stimulated with HA1-derived peptides, suggesting that IL-1b plays a crucial role in the enhancement of HA1-specific memory Th17 cell responses. Blocking IL-6 resulted in decreased Th17 cell responses. Taken together, the data obtained herein demonstrated that, in an IL-1 b-dependent manner, TLR2 ligand-mediated enhancement of HA1-specific Th17 cell responses are mainly due to the activation of memory Th17 cells. In FIG. 7E total CD4⁺ T cells were co-cultured with IFNDCs loaded with 1 ug/ml anti-hDectin-1-HA1 in the presence 40 ng/ml PG-LPS with indicated antibodies (10 g/ml of each) for seven days. CD4⁺ T cells were then restimulated with pep43 (donor #1), pep7 (donor #2), pep22 (donor #4), and pep22 (donor #5) for 36 h and IFNγ and IL-17 levels in the culture supernatants were measured.

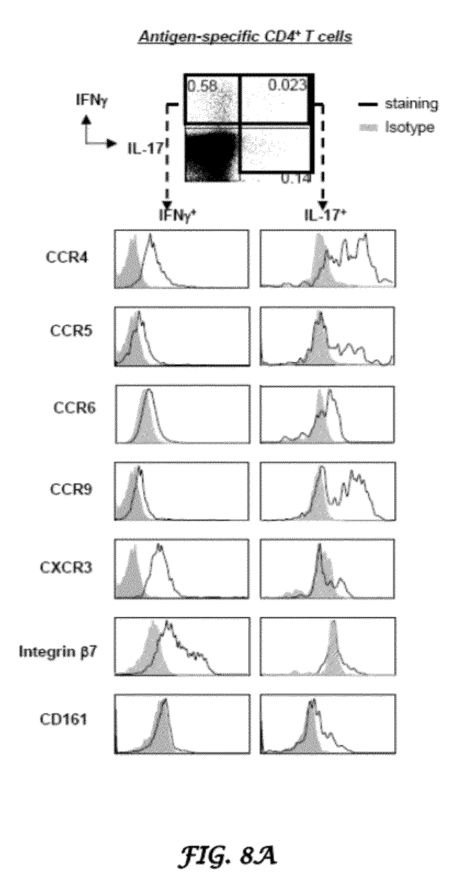

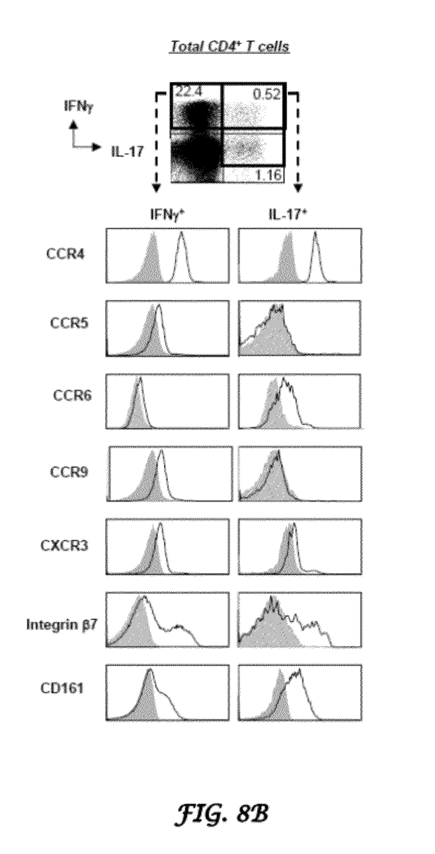

HA1-specific Th17 CD4⁺ T cells express high levels of CCR4, CCR6, and CCR9, but low levels of CD161 and 137 integrin. Phenotype of HA1-specific Th17 and Th1 cells expanded with anti-hDectin-1-HA1 was tested. Flow cytometry analysis shows that a large fraction of the HA1-specific Th17 cells express CCR4 and CCR6, whereas HA1-specific Th1 cells expressed CCR4 and CXCR3 (FIG. 8A). Compared to HA 1-specific Th1 cells, Th17 cells expressed lower levels of β7 integrin, but slightly higher levels of CD161 (33). Importantly, significant fractions of HA1-specific Th17 cells expressed high levels of CCR9. The inventors then compared the phenotype of HA1-specific CD4⁺ T cells vs. total CD4⁺ T cells in the same culture (FIG. 8B). Both HA1-specific and total Th1 cells expressed CCR4, CXCR3, and β7 integrin. However, a subset of only total Th1 cells expressed significant levels of CD161. Similarly, compared to total Th17 cells, HA1-specific Th17 cells expressed lower levels of CD161. The data obtained by the present inventors also show that only fractions of HA1-specific Th17 cells express high levels of CCR9 though the expression levels of CCR6 or CCR9 were not correlated to the capacity of IL-17 secretion (34). Addition of TLR2 ligands in the co-culture of DCs and CD4⁺ T cells did not enhance the expression levels of the chemokines receptors tested (data not shown).

The types of antigen-specific CD4+ T cells primed or boosted during infections and after vaccinations could determine the potency of protective immunity in the hosts (38). Th17 cells are now recognized as crucial components for protective immunity against infections of many microbial pathogens (4-18), including influenza viruses (39-42), and for the protection against subsequent infections. Thus, the proper activation and enhancement of pre-existing pathogen-specific Th17 cells is thought to be an efficient way to mount protective immunity. This study is the first demonstration that healthy individuals maintain pathogen (influenza)-specific Th17 cells and that such pathogen-specific memory Th17 cell responses can be further enhanced by targeting antigens to DCs via hDectin-1 in the presence of TLR2 ligands.

Dendritic cells (DCs) are the major antigen-presenting cells that can induce and control the quality of immune responses (43, 44). Thus, the study of Th17 cell responses elicited by DCs is more physiologically relevant than by T cells coupled with limited experimental conditions, such as APC-free cultures with anti-CD3/CD28 stimuli, exogenous cytokines, and neutralization of IFNg and IL-4. Delivering antigens to DC via a surface lectin, DEC205, has demonstrated an efficient way to elicit potent and broad spectrum antigen-specific T cell responses (28, 29). One such lectin-like receptors expressed on DCs, Dectin-1, is strongly associated with the induction and promotion of Th17 CD4+ T cells (10, 35, 45, 46). Signaling via Dectin-1 activates DCs to secrete IL-1b, IL-6, and IL-23 that contribute to the enhanced Th17 cell responses (10, 20, 47). Carter et al., also showed that antigens delivered to mouse DCs via Dectin-1 resulted in antigen-specific CD4+ T cell responses (48). The present inventors have previously reported that antigen targeting to human DCs via Dectin-1 using recombinant proteins of agonistic anti-hDectin-1 fused to antigens resulted in potent antigen-specific CD8+ T cell responses in vitro (Ni et al. 2010). Therefore, hDectin-1 expressed on DCs is considered to be a prominent target molecule to deliver antigens to DCs. In support of this, the strategy employed in this study, targeting HA1 to DCs via hDectin-1, allowed the inventors to characterize multiple HA1-derived peptide epitopes that have not been previously described.

Most importantly, antigen targeting to DCs via hDectin-1 permitted the inventors to detect pathogen (HA1 of influenza viruses)-specific memory Th17 cell responses in healthy individuals. It has not been easy to detect Th17 memory T cells specific for pathogen-derived peptides in vivo, and this was partly due to the low frequency of such Th17 cells in healthy hosts. A recent study showed that pathogen-specific Th17 cells are shorter-lived than Th1 cells in mice infected with Listeria monocytogene (27). Taking the advantages of the strategy described herein, targeting antigens to DCs via hDectin-1, the inventors first demonstrated that healthy individuals maintain influenza viral peptide epitope-specific memory Th17 cells. The agonistic property of anti-hDectin-1 fused to HA1 resulted in IL-23 induction from DCs, and this contributed to the amplification of HA 1-specific memory Th17 cell responses in vitro. Although IL-23 promoted Th17 cell responses, as previously described (33-35), IL-23 alone may not be sufficient to mount potent pathogen-specific Th17 cell responses.

The magnitudes of HA1-specific memory Th17 cell responses were not correlated with the magnitudes of other types of HA 1-specific CD4+ T cell responses. However, there was a correlation between the magnitudes of HA1-specific Th1 cell responses and those of HA1-specific Th2 cell responses. Additionally, the magnitudes of Th17 cell responses observed were highly variable among individuals and among peptide epitopes. These findings, the presence of HA1-specific memory Th17 cells in healthy individuals, are of fundamental importance because of the potential to promote such memory Th17 cell responses in healthy individuals.

The roles of TLR2 ligands in the expansion of Th17 CD4+ T cell responses are not clearly elucidated. TLR2 deficiency results in increased Th17 immunity associated with diminished expansion of regulatory T cells (49). It is also known that TLR2 promote regulatory T cell responses that inhibited autoimmunity in mice (50). In contrast, TLR2 engagement on DCs promotes influenza viral specific memory CD4+ T cell responses (41). In addition, activation of hDCs via Dectin-1 and TLR2 resulted in enhanced Th17 responses (51, 52). Those discrepancy could be dependent on several factors, such as the strength of signals delivered to DCs via TLR2, integration of different signals delivered to DCs at the same time, subsets of DCs or T cells (memory vs. naïve), and distinct specie differentiation (i.e. human vs. non-human models). However, the role of TLR2 ligands in the enhancement of HA1-specific memory Th17 responses was solid and generic. TLR2 ligands were capable to enhance memory Th17 responses and Th1 in a less extent in all healthy donors tested. A previous study (53) showed that freshly isolated circulating human Th17 cells secrete IL-17 alone or with IL2, but those activated by DCs co-express IL-17 and IFNg. In combination with anti-hDectin-1-HA1, TLR2 ligands did not significantly enhance HA1-specific IL-10-producing CD4+ T cell responses. E. coli LPS could also enhance HA1-specific Th17 cell responses, but it also enhanced IL-10-producing CD4+ T cell responses.

It is also important to note that the majority of HA1-specific Th17 cell responses were not the results of priming HA1-specific T cells, but the results of the activation of memory CD4+ T cells. IL-23 and IL-1β secreted by DCs enhanced HA 1-specific Th17 cell responses, but did not result in the induction of HA1-specific Th17 cells in vitro. While testing HA1-specific T cell responses, the inventors also assessed the allogeneic naïve CD4+ T cell responses induced by DCs, as many studies employ allogeneic systems to test the types of T cell responses induced in vitro. Indeed, the inventors observed the induction of allogeneic Th17 cell responses, which were further enhanced by activating DCs with anti-hDectin-1 mAb or curdlan, a fungal b-glucan (data not shown). The disparity observed between allogeneic T cells and antigen-specific T cells needs to be considered carefully, particularly when the induction of Th17 cell responses are assessed. The findings presented herein suggest that other factors, including signals from other immune cells and the strength of signaling via T cell receptors, are involved in the induction of pathogen-specific Th17 cells in vivo.

It is contemplated that any embodiment discussed in this specification can be implemented with respect to any method, kit, reagent or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C or combinations thereof” is intended to include at least one of: A, B, C, AB, AC, BC or ABC, and if order is important in a particular context, also BA, CA, CB, CBA, BCA, ACB, BAC or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

United States Patent Application No. 2010/0166784: Method and Compositions for Modulating Th17 Cell Development.
United States Patent Application No. 2008/0233140: Therapeutic Applications of Activation of Human Antigen-Presenting Cells Through Dectin-1.
1. Miossec, P., T. Korn, and V. K. Kuchroo. 2009. Interleukin-17 and type 17 helper T cells. N Engl J Med 361:888-898.
2. Fouser, L. A., J. F. Wright, K. Dunussi-Joannopoulos, and M. Collins. 2008. Th17 cytokines and their emerging roles in inflammation and autoimmunity. Immunol Rev 226:87-102.
3. Mills, K. H. 2008. Induction, function and regulation of IL-17-producing T cells. Eur J Immunol 38:2636-2649.
4. O'Connor, W., L. A. Zenewicz, and R. A. Flavell. The dual nature of TH17 cells: shifting the focus to function. Nat Immunol 11:471-476.
5. Zenaro, E., M. Donini, and S. Dusi. 2009. Induction of Th1/Th17 immune response by Mycobacterium tuberculosis: role of dectin-1, Mannose Receptor, and DC-SIGN. J Leukoc Biol 86:1393-1401.
6. Khader, S. A., and A. M. Cooper. 2008. IL-23 and IL-17 in tuberculosis. Cytokine 41:79-83.
7. Schulz, S. M., G. Kohler, N. Schutze, J. Knauer, R. K. Straubinger, A. A. Chackerian, E. Witte, K. Wolk, R. Sabat, Y. Iwakura, C. Holscher, U. Muller, R. A. Kastelein, and G. Alber. 2008. Protective immunity to systemic infection with attenuated Salmonella enterica serovar enteritidis in the absence of IL-12 is associated with IL-23-dependent IL-22, but not IL-17. J Immunol 181:7891-7901.
8. Conti, H. R., F. Shen, N. Nayyar, E. Stocum, J. N. Sun, M. J. Lindemann, A. W. Ho, J. H. Hai, J. J. Yu, J. W. Jung, S. G. Filler, P. Masso-Welch, M. Edgerton, and S. L. Gaffen. 2009. Th17 cells and IL-17 receptor signaling are essential for mucosal host defense against oral candidiasis. J Exp Med 206:299-311.
9. Acosta-Rodriguez, E. V., L. Rivino, J. Geginat, D. Jarrossay, M. Gattorno, A. Lanzavecchia, F. Sallusto, and G. Napolitani. 2007. Surface phenotype and antigenic specificity of human interleukin 17-producing T helper memory cells. Nat Immunol 8:639-646.
10. Leibundgut-Landmann, S., O. Gross, M. J. Robinson, F. Osorio, E. C. Slack, S. V. Tsoni, E. Schweighoffer, V. Tybulewicz, G. D. Brown, J. Ruland, and E. S. C. Reis. 2007. Syk- and CARD9-dependent coupling of innate immunity to the induction of T helper cells that produce interleukin 17. Nat Immunol 8:630-638.
11. Milner, J. D., J. M. Brenchley, A. Laurence, A. F. Freeman, B. J. Hill, K. M. Elias, Y. Kanno, C. Spalding, H. Z. Elloumi, M. L. Paulson, J. Davis, A. Hsu, A. I. Asher, J. O'Shea, S. M. Holland, W. E. Paul, and D. C. Douek. 2008. Impaired T(H)17 cell differentiation in subjects with autosomal dominant hyper-IgE syndrome. Nature 452:773-776.
12. Williman, J., E. Lockhart, L. Slobbe, G. Buchan, and M. Baird. 2006. The use of Th1 cytokines, IL-12 and IL-23, to modulate the immune response raised to a DNA vaccine delivered by gene gun. Vaccine 24:4471-4474.
13. Kohyama, S., S. Ohno, A. Isoda, O. Moriya, M. L. Belladonna, H. Hayashi, Y. Iwakura, T. Yoshimoto, T. Akatsuka, and M. Matsui. 2007. IL-23 enhances host defense against vaccinia virus infection via a mechanism partly involving IL-17. J Immunol 179:3917-3925.
14. Smiley, K. L., M. M. McNeal, M. Basu, A. H. Choi, J. D. Clements, and R. L. Ward. 2007. Association of gamma interferon and interleukin-17 production in intestinal CD4+ T cells with protection against rotavirus shedding in mice intranasally immunized with VP6 and the adjuvant LT(R192G). J Virol 81:3740-3748.
15. Kelly, M. N., J. K. Kolls, K. Happel, J. D. Schwartzman, P. Schwarzenberger, C. Combe, M. Moretto, and I. A. Khan. 2005. Interleukin-17/interleukin-17 receptor-mediated signaling is important for generation of an optimal polymorphonuclear response against Toxoplasma gondii infection. Infect Immun 73:617-621.
16. Huang, W., L. Na, P. L. Fidel, and P. Schwarzenberger. 2004. Requirement of interleukin-17A for systemic anti-Candida albicans host defense in mice. J Infect Dis 190:624-631.
17. Khader, S. A., G. K. Bell, J. E. Pearl, J. J. Fountain, J. Rangel-Moreno, G. E. Cilley, F. Shen, S. M. Eaton, S. L. Gaffen, S. L. Swain, R. M. Locksley, L. Haynes, T. D. Randall, and A. M. Cooper. 2007. IL-23 and IL-17 in the establishment of protective pulmonary CD4+ T cell responses after vaccination and during Mycobacterium tuberculosis challenge. Nat Immunol 8:369-377.
18. Pitta, M. G., A. Romano, S. Cabantous, S. Henri, A. Hammad, B. Kouriba, L. Argiro, M. el Kheir, B. Bucheton, C. Mary, S. H. El-Safi, and A. Dessein. 2009. IL-17 and IL-22 are associated with protection against human kala azar caused by Leishmania donovani. J Clin Invest 119:2379-2387.
19. Korn, T., E. Bettelli, M. Oukka, and V. K. Kuchroo. 2009. IL-17 and Th17 Cells. Annu Rev Immunol 27:485-517.
20. Acosta-Rodriguez, E. V., G. Napolitani, A. Lanzavecchia, and F. Sallusto. 2007. Interleukins 1beta and 6 but not transforming growth factor-beta are essential for the differentiation of interleukin 17-producing human T helper cells. Nat Immunol 8:942-949.
21. Wilson, N. J., K. Boniface, J. R. Chan, B. S. McKenzie, W. M. Blumenschein, J. D. Mattson, B. Basham, K. Smith, T. Chen, F. Morel, J. C. Lecron, R. A. Kastelein, D. J. Cua, T. K. McClanahan, E. P. Bowman, and R. de Waal Malefyt. 2007. Development, cytokine profile and function of human interleukin 17-producing helper T cells. Nat Immunol 8:950-957.
22. Manel, N., D. Unutmaz, and D. R. Littman. 2008. The differentiation of human T(H)-17 cells requires transforming growth factor-beta and induction of the nuclear receptor RORgammat. Nat Immunol 9:641-649.
23. Volpe, E., N. Servant, R. Zollinger, S. I. Bogiatzi, P. Hupe, E. Barillot, and V. Soumelis. 2008. A critical function for transforming growth factor-beta, interleukin 23 and proinflammatory cytokines in driving and modulating human T(H)-17 responses. Nat Immunol 9:650-657.
24. Yang, L., D. E. Anderson, C. Baecher-Allan, W. D. Hastings, E. Bettelli, M. Oukka, V. K. Kuchroo, and D. A. Hafler. 2008. IL-21 and TGF-beta are required for differentiation of human T(H)17 cells. Nature 454:350-352.
25. van Beelen, A. J., Z. Zelinkova, E. W. Taanman-Kueter, F. J. Muller, D. W. Hommes, S. A. Zaat, M. L. Kapsenberg, and E. C. de Jong. 2007. Stimulation of the intracellular bacterial sensor NOD2 programs dendritic cells to promote interleukin-17 production in human memory T cells. Immunity 27:660-669.
26. Liu, H., and C. Rohowsky-Kochan. 2008. Regulation of IL-17 in human CCR6+ effector memory T cells. J Immunol 180:7948-7957.
27. Pepper, M., J. L. Linehan, A. J. Pagan, T. Zell, T. Dileepan, P. P. Cleary, and M. K. Jenkins. Different routes of bacterial infection induce long-lived TH1 memory cells and short-lived TH17 cells. Nat Immunol 11:83-89.
28. Bonifaz, L., D. Bonnyay, K. Mahnke, M. Rivera, M. C. Nussenzweig, and R. M. Steinman. 2002. Efficient targeting of protein antigen to the dendritic cell receptor DEC-205 in the steady state leads to antigen presentation on major histocompatibility complex class I products and peripheral CD8+ T cell tolerance. J Exp Med 196:1627-1638.
29. Boscardin, S. B., J. C. Hafalla, R. F. Masilamani, A. O. Kamphorst, H. A. Zebroski, U. Rai, A. Morrot, F. Zavala, R. M. Steinman, R. S, Nussenzweig, and M. C. Nussenzweig. 2006. Antigen targeting to dendritic cells elicits long-lived T cell help for antibody responses. J Exp Med 203:599-606.
30. Reddy, M. P., C. A. Kinney, M. A. Chaikin, A. Payne, J. Fishman-Lobell, P. Tsui, P. R. Dal Monte, M. L. Doyle, M. R. Brigham-Burke, D. Anderson, M. Reff, R. Newman, N. Hanna, R. W. Sweet, and A. Truneh. 2000. Elimination of Fc receptor-dependent effector functions of a modified IgG4 monoclonal antibody to human CD4. J Immunol 164:1925-1933.
31. Diebold, S. S., M. Montoya, H. Unger, L. Alexopoulou, P. Roy, L. E. Haswell, A. Al-Shamkhani, R. Flavell, P. Borrow, and C. Reis e Sousa. 2003. Viral infection switches non-plasmacytoid dendritic cells into high interferon producers. Nature 424:324-328.
32. Blanco, P., A. K. Palucka, M. Gill, V. Pascual, and J. Banchereau. 2001. Induction of dendritic cell differentiation by IFN-alpha in systemic lupus erythematosus. Science 294:1540-1543.
33. Oppmann, B., R. Lesley, B. Blom, J. C. Timans, Y. Xu, B. Hunte, F. Vega, N. Yu, J. Wang, K. Singh, F. Zonin, E. Vaisberg, T. Churakova, M. Liu, D. Gorman, J. Wagner, S. Zurawski, Y. Liu, J. S. Abrams, K. W. Moore, D. Rennick, R. de Waal-Malefyt, C. Hannum, J. F. Bazan, and R. A. Kastelein. 2000. Novel p19 protein engages IL-12p40 to form a cytokine, IL-23, with biological activities similar as well as distinct from IL-12. Immunity 13:715-725.
34. Piskin, G., R. M. Sylva-Steenland, J. D. Bos, and M. B. Teunissen. 2006. In vitro and in situ expression of IL-23 by keratinocytes in healthy skin and psoriasis lesions: enhanced expression in psoriatic skin. J Immunol 176:1908-1915.
35. Carmona, E. M., R. Vassallo, Z. Vuk-Pavlovic, J. E. Standing, T. J. Kottom, and A. H. Limper. 2006. Pneumocystis cell wall beta-glucans induce dendritic cell costimulatory molecule expression and inflammatory activation through a Fas-Fas ligand mechanism. J Immunol 177:459-467.
36. Darveau, R. P., T. T. Pham, K. Lemley, R. A. Reife, B. W. Bainbridge, S. R. Coats, W. N. Howald, S. S. Way, and A. M. Hajjar. 2004. Porphyromonas gingivalis lipopolysaccharide contains multiple lipid A species that functionally interact with both toll-like receptors 2 and 4. Infect Immun 72:5041-5051.
37. Burns, E., T. Eliyahu, S. Uematsu, S. Akira, and G. Nussbaum. TLR2-dependent inflammatory response to Porphyromonas gingivalis is MyD88 independent, whereas MyD88 is required to clear infection. J Immunol 184:1455-1462.
38. Murphy, K. M., and S. L. Reiner. 2002. The lineage decisions of helper T cells. Nat Rev Immunol 2:933-944.
39. Bermejo-Martin, J. F., R. Ortiz de Lejarazu, T. Pumarola, J. Rello, R. Almansa, P. Ramirez, I. Martin-Loeches, D. Varillas, M. C. Gallegos, C. Seron, D. Micheloud, J. M. Gomez, A. Tenorio-Abreu, M. J. Ramos, M. L. Molina, S. Huidobro, E. Sanchez, M. Gordon, V. Fernandez, A. Del Castillo, M. A. Marcos, B. Villanueva, C. J. Lopez, M. Rodriguez-Dominguez, J. C. Galan, R. Canton, A. Lietor, S. Rojo, J. M. Eiros, C. Hinojosa, I. Gonzalez, N. Torner, D. Banner, A. Leon, P. Cuesta, T. Rowe, and D. J. Kelvin. 2009. Th1 and Th17 hypercytokinemia as early host response signature in severe pandemic influenza. Crit. Care 13:R201.
40. McKinstry, K. K., T. M. Strutt, A. Buck, J. D. Curtis, J. P. Dibble, G. Huston, M. Tighe, H. Hamada, S. Sell, R. W. Dutton, and S. L. Swain. 2009. IL-10 deficiency unleashes an influenza-specific Th17 response and enhances survival against high-dose challenge. J Immunol 182:7353-7363.
41. Chandran, S. S., D. Verhoeven, J. R. Teijaro, M. J. Fenton, and D. L. Farber. 2009. TLR2 engagement on dendritic cells promotes high frequency effector and memory CD4 T cell responses. J Immunol 183:7832-7841.
42. Hamada, H., L. Garcia-Hernandez Mde, J. B. Reome, S. K. Misra, T. M. Strutt, K. K. McKinstry, A. M. Cooper, S. L. Swain, and R. W. Dutton. 2009. Tc17, a unique subset of CD8 T cells that can protect against lethal influenza challenge. J Immunol 182:3469-3481.
43. Dillon, S., A. Agrawal, T. Van Dyke, G. Landreth, L. McCauley, A. Koh, C. Maliszewski, S. Akira, and B. Pulendran. 2004. A Toll-like receptor 2 ligand stimulates Th2 responses in vivo, via induction of extracellular signal-regulated kinase mitogen-activated protein kinase and c-Fos in dendritic cells. J Immunol 172:4733-4743.
44. Banchereau, J., B. Pulendran, R. Steinman, and K. Palucka. 2000. Will the making of plasmacytoid dendritic cells in vitro help unravel their mysteries? J Exp Med 192:F39-44.
45. Weck, M. M., S. Appel, D. Werth, C. Sinzger, A. Bringmann, F. Grunebach, and P. Brossart. 2008. hDectin-1 is involved in uptake and cross-presentation of cellular antigens. Blood 111:4264-4272.
46. Brown, G. D. 2006. Dectin-1: a signalling non-TLR pattern-recognition receptor. Nat Rev Immunol 6:33-43.
47. Gross, O., A. Gewies, K. Finger, M. Schafer, T. Sparwasser, C. Peschel, I. Forster, and J. Ruland. 2006. Card9 controls a non-TLR signalling pathway for innate anti-fungal immunity. Nature 442:651-656.
48. Carter, R. W., C. Thompson, D. M. Reid, S. Y. Wong, and D. F. Tough. 2006. Preferential induction of CD4+ T cell responses through in vivo targeting of antigen to dendritic cell-associated C-type lectin-1. J Immunol 177:2276-2284.
49. Loures, F. V., A. Pina, M. Felonato, and V. L. Calich. 2009. TLR2 is a negative regulator of Th17 cells and tissue pathology in a pulmonary model of fungal infection. J Immunol 183:1279-1290.
50. Manicassamy, S., R. Ravindran, J. Deng, H. Oluoch, T. L. Denning, S. P. Kasturi, K. M. Rosenthal, B. D. Evavold, and B. Pulendran. 2009. Toll-like receptor 2-dependent induction of vitamin A-metabolizing enzymes in dendritic cells promotes T regulatory responses and inhibits autoimmunity. Nat Med 15:401-409.
51. Duraisingham, S. S., J. Hornig, F. Gotch, and S. Patterson. 2009. TLR-stimulated CD34 stem cell-derived human skin-like and monocyte-derived dendritic cells fail to induce Th17 polarization of naive T cells but do stimulate Th1 and Th17 memory responses. J Immunol 183:2242-2251.
52. Aliahmadi, E., R. Gramlich, A. Grutzkau, M. Hitzler, M. Kruger, R. Baumgrass, M. Schreiner, B. Wittig, R. Wanner, and M. Peiser. 2009. TLR2-activated human langerhans cells promote Th17 polarization via IL-1beta, TGF-beta and IL-23. Eur J Immunol 39:1221-1230.
53. Dhodapkar, K. M., S. Barbuto, P. Matthews, A. Kukreja, A. Mazumder, D. Vesole, S. Jagannath, and M. V. Dhodapkar. 2008. Dendritic cells mediate the induction of polyfunctional human IL17-producing cells (Th17-1 cells) enriched in the bone marrow of patients with myeloma. Blood 112:2878-2885.

What is claimed is:

1. A method for enhancing antigen-specific T cell responses in a Dectin-1-expressing antigen presenting cell (APC) comprising the steps of:
loading the APC with an anti-Dectin-1-specific antibody or binding fragment thereof conjugated or fused with one or more antigens;
contacting the antigen-loaded APC with T cells; and
isolating T cells that proliferate when contacted with the antigen-loaded APC, wherein the antigen-specific T cell response is enhanced to secrete IL-23.

2. The method of claim 1, wherein the one or more antigens comprise bacterial, fungal or viral antigens.

3. The method of claim 1, wherein the antigen is a HA1 subunit of an influenza virus.

4. The method of claim 1, wherein the method reduces Th2 cell responses.

5. The method of claim 1, wherein the method enhances Th17 and Th1.

6. The method of claim 1, wherein the composition optionally comprises one or more TLR2 ligands.

7. The method of claim 6, wherein the one or more TLR2 ligands comprise heat-killed bacteria, lipoglycans, lipopolysaccharide, lipoteichoic acids, peptidoglycans, synthetic lipoproteins, zymosan or combinations and modifications thereof.

8. The method of claim 6, wherein the TLR2 ligand comprises lipopolysaccharides comprising P. gingivalis LPS or E. coli LPS.

9. The method of claim 8, wherein the method increases secretion of IL-1β, IL-6, and IL-23 thereby leading to an enhanced Th17 response.

10. The method of claim 8, wherein the method reduces Th2 cell responses.

11. An influenza vaccine composition for prophylaxis, treatment, amelioration of symptoms or combinations thereof comprising:
an anti-Dectin-1-specific antibody or binding fragment thereof fused with a HA1 subunit of an influenza virus; and
one or more optional pharmaceutically acceptable excipients or adjuvants.

12. The composition of claim 11, wherein the composition enhances Th17 and Th1 responses by a secretion of IL-23.

13. The composition of claim 11, wherein the composition reduces Th2 cell responses.

14. The composition of claim 11, wherein the composition is administered by an oral route, a parenteral route or an intra-nasal route.

15. The composition of claim 11, wherein the composition optionally comprises one or more TLR2 ligands.

16. The composition of claim 11, wherein the one or more TLR2 ligands comprise heat-killed bacteria, lipoglycans, lipopolysaccharide, lipoteichoic acids, peptidoglycans, synthetic lipoproteins, zymosan or combinations and modifications thereof.

17. The composition of claim 16, wherein the TLR2 ligand comprises lipopolysaccharides comprising P. gingivalis LPS or E. coli LPS.

18. The composition of claim 17, wherein the composition increases secretion of IL-1β, IL-6, and IL-23 thereby leading to an enhanced Th17 response.

19. The composition of claim 17, wherein the composition reduces Th2 cell responses.

20. A method for treating, prophylaxis or amelioration of symptoms of influenza in a human subject comprising the steps of:
identifying the subject in need of the treatment, prophylaxis or amelioration of symptoms of the influenza; and
administering a therapeutically effective amount of a pharmaceutical composition or a vaccine comprising an anti-Dectin-1-specific antibody or binding fragment thereof fused with a HA1 subunit of an influenza virus and one or more optional pharmaceutically acceptable excipients or adjuvants in an amount sufficient for the treatment, prophylaxis or amelioration of the symptoms of the influenza.

21. The method of claim 20, wherein the composition is administered by an oral route, a parenteral route or an intra-nasal route.

22. The method of claim 20, wherein the composition optionally comprises one or more TLR2 ligands.

23. The method of claim 22, wherein the one or more TLR2 ligands comprise heat-killed bacteria, lipoglycans, lipopolysaccharide, lipoteichoic acids, peptidoglycans, synthetic lipoproteins, zymosan or combinations and modifications thereof.

24. The method of claim 22, wherein the TLR2 ligand comprises lipopolysaccharides comprising P. gingivalis LPS or E. coli LPS.

25. A composition for enhancing antigen-specific T cell responses in a Dectin-1-expressing antigen presenting cell (APC) comprising an anti-Dectin-1-specific antibody or binding fragment thereof fused with one or more antigens.

26. The composition of claim 25, wherein the APC comprises an isolated dendritic cell (DC), a peripheral blood mononuclear cell (PBMC), a monocyte, a B cell, a myeloid dendritic cell or combinations thereof.

27. The composition of claim 25, wherein the APC comprises an isolated dendritic cell (DC), a peripheral blood mononuclear cell, a monocyte, a B cell, a myeloid dendritic cell or combinations thereof that have been cultured in vitro with GM-CSF and IL-4, IFNα, antigen, and combinations thereof.

28. The composition of claim 25, wherein the one or more antigens comprise bacterial, fungal or viral antigens.

29. The composition of claim 25, wherein the antigen is a HA1 subunit of an influenza virus.

30. The composition of claim 25, wherein the composition optionally comprises one or more TLR2 ligands.

31. The composition of claim 25, wherein the one or more TLR2 ligands comprise heat-killed bacteria, lipoglycans, lipopolysaccharide, lipoteichoic acids, peptidoglycans, synthetic lipoproteins, zymosan or combinations and modifications thereof.

32. The composition of claim 31, wherein the TLR2 ligand comprises lipopolysaccharides comprising P. gingivalis LPS or E. coli LPS.

33. The composition of claim 31, wherein the composition results in a proliferation of CD4⁺ T cells.

34. The composition of claim 33, wherein the CD4⁺ T secrete one or more cytokines selected from the group consisting of IFNγ, IL-13, IL-10, IL-17, and IL-21.

35. The composition of claim 30, wherein the composition enhances Th17 and Th1 responses by a secretion of IL-23.

36. The composition of claim 30, wherein the composition reduces Th2 cell responses.

37. The composition of claim 30, wherein the composition increases secretion of IL-1β, IL-6, and IL-23 thereby leading to an enhanced Th-17 response.
